(19) United States
(12) Patent Application Publication
(10) Pub. No.: US 2005/0084841 A1
(43) Pub. Date: Apr. 21, 2005

(54) Isle gene and its association with osteoarthritis and other bone and cartilage disorders expression products derived therefrom and uses thereof

(76) Inventors: Orit Segev, Rehovot (IL); Elena Feinstein, Rehovot (IL)

Correspondence Address: COOPER & DUNHAM, LLP, 1185 AVENUE OF THE AMERICAS, NEW YORK, NY, 10036, US

(21) Appl. No.: 10/503,141

(22) PCT Filed: Jan. 29, 2003
(86) PCT No.: PCT/US03/02666

Related U.S. Application Data
(63) Continuation-in-part of application No. 60/352,655, filed on Jan. 29, 2002.
(63) Continuation-in-part of application No. 60/352,658, filed on Jan. 29, 2002.

(51) Int. Cl. C12Q 1/68; C12Q 1/00

(52) U.S. Cl. 435/4; 435/6

(57) ABSTRACT

The disclosure relates to the ISLR gene, and functional equivalents thereof, such as those from humans and from mice, probes therefor, tests to identify such genes, expression products of such genes, uses for such genes and expression products, e.g., in diagnosis (for instance, risk determination), treatment, prevention, or control of osteoarthritis or rheumatoid arthritis or osteoporosis or other bone disorders or conditions or factors or processes which lead to these disorders or conditions; and to diagnosis, treatment, prevention, or control methods or processes, as well as compositions therefor and methods or processes for making and using such compositions, and receptors therefor and methods or processes for obtaining and using such receptors.

This invention is a continuation-in-part and claims the benefit of U.S. Provisional Application No. 60/352,655, filed Jan. 29, 2002, and U.S. Provisional Application No. 60/352,658, filed Jan. 29, 2002, the contents of which are hereby incorporated by reference into this application.

### FIELD OF THE INVENTION

This invention relates to IL1-β-induced genes, in particular ISLR. This invention also relates to functional equivalents of ISLR and probes therefor. This invention further relates to tests to identify such IL1-β-induced genes, expression products of such genes, uses for such genes and expression products, e.g., in diagnosis (for instance, risk determination), treatment, prevention or control of osteoarthritis (OA) and cartilage damage associated therewith, and of rheumatoid arthritis (RA) and of osteopenia (low bone density) that includes osteoporosis and periodontosis and treatment of symptoms thereof, and a method for promotion of bone building and bone repair that includes bone fracture healing and bone elongation. This invention further relates to diagnosis, treatment, prevention or control of processes which lead to the above diseases and conditions as well as to compositions therefor and methods or processes for making and using such compositions, and receptors for such expression products and methods or processes for obtaining and using such receptors.

The invention also relates to the involvement of genes, in particular ISLR, with the development of the above diseases and conditions. More particularly, the invention relates to methods of treatment, compositions and the use of specific modulators in the treatment of the above diseases and conditions and in cartilage rehabilitation.

### BACKGROUND OF THE INVENTION

Osteoporosis (OP)

Bone is composed of a collagen-rich organic matrix impregnated with mineral, largely calcium and phosphate. Two major forms of bone exist, compact cortical bone forms the external envelopes of the skeleton, and trabecular or medullary bone forms plates that traverse the internal cavities of the skeleton. The responses of these two forms to metabolic influences and their susceptibility to fracture differ.

Bone undergoes continuous remodeling (e.g., turnover, renewal) throughout life. Mechanical and electrical forces, hormones and local regulatory factors influence remodeling. Bone is renewed by two opposing activities that are coupled in time and space (Parfitt (1979), Calcified. Tissue. Int. 28:1-5). These activities, resorption and formation, are contained within a temporary anatomic structure known as a bone-remodeling unit (Parfitt (1981), Resident and Staff Physician December:60-72). Within a given bone-remodeling unit, old bone is resorbed by osteoclasts. The resorbed cavity created by osteoclasts is subsequently filled with new bone by osteoblasts, synthesizing bone organic matrix.

Peak bone mass is mainly genetically determined, although dietary factors and physical activity can have positive effects. Peak bone mass is attained at the point when skeletal growth ceases, after which time bone loss begins.

In contrast to the positive balance that occurs during growth, in OP the resorbed cavity is not completely refilled by bone (Parfitt (1988), Osteoporosis: Etiology, Diagnosis, and Management (Riggs and Melton, eds.) Raven Press, New York, pp. 74-93). OP, or porous bone, is a progressive and chronic disease characterized by low bone mass and structural deterioration of bone tissue, leading to bone fragility and an increased susceptibility to fracture of the hip, spine, and wrist (diminishing bone strength).

Bone loss occurs without symptoms. The Consensus Development Conference ((1993) Am. J. Med. 94:646-650) defined OP as “a systemic skeletal disease characterized by low bone mass and microarchitectural deterioration of bone tissue, with a consequent increase in bone fragility and susceptibility to fracture.”

Common types of OP include postmenopausal OP and senile OP, the latter of which generally occurs in later life, e.g., 70+ years (see e.g., U.S. Pat. No. 5,691,153). OP is estimated to affect more than 25 million people in the United States (Rosen (1997) Calcified. Tissue Int. 60:225-228) and at least one estimate asserts that OP affects 1 in 3 women (Keen et al. (1997) Drugs Aging 11:333-337). Moreover, life expectancy has increased and, in the Western world, 17% of women are now over 50 years of age: a woman can expect to live one third of her life after menopause. Thus, some estimate that 1 out of every 2 women and 1 out of every 5 men will eventually develop OP, and that 75 million people in the U.S., Japan and Europe have OP. The World Summit of Osteoporosis Societies estimates that more than 200 million people worldwide are afflicted with the disease. Another estimate is that 30 million Americans are at risk for OP, the most common among bone diseases, and that there are probably 100 million people similarly at risk worldwide (Melton (1995) Bone Min. Res. 10:175). These numbers are growing as the proportion of the elderly in the world population increases. The actual incidence of the disease is difficult to estimate since the condition is often asymptomatic until a bone fracture occurs. It is believed that there are over 1.5 million OP-associated bone fractures per year in the U.S. alone. Of these, 300,000 are hip fractures that usually require hospitalization and surgery and may result in lengthy or permanent disability or even death. See a minireview by Spangler et al. “The Genetic Component of Osteoporosis” (1997)Cambridge Scientific Abstracts”.

OP is a major health problem in virtually all societies (Eisman (1996) Curr.Opin.Genet. Dev 6(3):361-365; Wark (1996); Maturitas 23:193-207; and U.S. Pat. No. 5,834,200). There is a 20-30% mortality rate related to hip fractures in elderly women (U.S. Pat. No. 5,691,153); and such a patient with a hip fracture has a 10-15% greater chance of dying than others of the same age. Furthermore, although men suffer fewer hip injuries than do women, men are 25% more likely than women to die within one year of the injury (see Spangler et al., supra). Also, about 20% of the patients who lived independently before a hip fracture remain confined in a long-term health care facility one year after such a fracture. The treatment of OP and related fractures costs over $10 billion annually.

OP treatment helps check further bone loss and fractures. Common therapeutics include HRT (hormone replacement therapy), bisphosphonates, e.g., alendronate (Fosamax), estrogen and estrogen receptor modulators, progestin, calcitonin, and vitamin D. While there may be numerous factors that determine whether any particular person will develop OP, a major step towards prevention, control or treatment of OP is determining whether one is at risk for OP. Genetic factors also play an important role in the pathogenesis of OP (Ralston (1997)QJM-Monthly Journal of the Association of Physicians (U.K.) 90/4,247-251; Keen et al. (1997) Drugs Aging (New Zealand) 11(5) :333-337; Eisman (1996) supra; Rosen (1997), Calcif. Tissue Int, 60(3):225-228; Cole and Rubin (1998) Presentation No.SAcole0195, presented at INABIS'98-5^{th} Internet World Congress on Biomedical Sciences at McMaster University, Canada, December 7-16.; Johnston et al. (1995) Bone 17(2 Suppl)19S-22S; Gong et al. (1996) Am. J. Hum. Genet. 59:146-151; and Wasnich (1996) Bone 18(3 Suppl):179S-183S). Some attribute 50-60% of total bone variation (bone mineral density: “BMD”), depending upon the bone area, to genetic effects (Livshits et al. (1996) Hum. Biol. 68:540-554). Others attribute up to 90% of such variance in bone mineral density to genetic effects.

Studies of family histories, twin studies, and racial factors have shown that there may be a predisposition for OP (Jouanny et al. (1995) Arthritis Rheum. 38:61-67; Garnero et al. (1996) J. Clin. Endrocrinol. Metab. 81:140-146; Cummings (1996) Bone 18(3 Suppl):165S-167S; and Lonzer et al. (1996) Clin. Pediatr. 35:185-189). Several candidate genes may be involved in this most probably multigenic process.

Cytokines are powerful regulators of bone resorption and formation under control of estrogen/testosterone, parathyroid hormone and 1,25(OH)2D3. Some cytokines primarily enhance osteoclastic bone resorption e.g., IL-1 (interleukin-1), TNF (tumor necrosis factor) and IL-6 (interleukin-6), while others primarily stimulate bone formation, e.g., TGF-(transforming growth factor-), IGF (insulin-like growth factor) and PDGF (platelet derived growth factor).

There exists a need for clinical and epidemiological research for the prevention and treatment of OP for gaining greater knowledge of factors controlling bone cell activity and regulation of bone mineral and matrix formation and remodeling.

Despite recent successes with drugs that inhibit bone resorption, there is a clear need for specific anabolic agents that will considerably increase bone formation in people who have already suffered substantial bone loss. There are no such drugs currently approved.

Bone develops via a number of processes. Mesenchymal cells can differentiate directly into bone, as occurs in the flat bones of the craniofacial skeleton; this process is termed intramembraneous ossification. Alternatively, cartilage provides a template for bone morphogenesis, as occurs in the majority of human bones. The cartilage template is replaced by bone in a process known as endochondral ossification (Reddi (1981) Collagen Rel. Res. 1:209-226). Bone is also continuously modeled during growth and development and remodeled throughout the life of the organism in response to physical and chemical signals. Development and maintenance of cartilage and bone tissue during embryogenesis and throughout the lifetime of vertebrates is very complex. It is widely accepted that a multitude of factors, from systemic hormones to local regulatory factors such as the members of the TGF-superfamily, cytokines and prostaglandins, act in concert to regulate the continuous processes of bone formation and bone resorption. Disturbance of the balance between osteoblastic bone deposition and osteoclastic bone resorption is responsible for many skeletal diseases.

Diseases of bone loss are a major public health problem, especially for women in all Western communities. The most common cause of osteopenia is OP; other causes include osteomalacia and bone disease related to hyperparathyroidism. Osteopenia has been defined as the appearance of decreased bone mineral content on radiography, but the term more appropriately refers to a phase in the continuum from decreased bone mass to fractures and infirmity.

Mechanical stimulation induces new bone formation in vivo and increases osteoblastic differentiation and metabolic activity in culture. Mechanotransduction in bone tissue involves several steps: 1) mechanochemical transduction of the signal, 2) cell-to-cell signaling, and 3) increased number and activity of osteoblasts. Cell-to-cell signaling after mechanical stimulus involves prostaglandins, especially those produced by COX-2, and nitric oxide. Prostaglandins induce new bone formation by promoting both proliferation and differentiation of osteoprogenitor cells.

Osteoarthritis (OA)

OA is a common, debilitating, costly, and currently incurable disease. Novel approaches to therapy are clearly required. The disease is characterized by abnormal functioning of chondrocytes, their terminal differentiation and initiation of osteogenesis within articular cartilage tissue, and breakdown of normal cartilage matrix. Three categories of genes are potential candidates as targets for therapeutic intervention: (i) genes, the products of which are involved in chondrogenesis and osteogenesis (starting from the common progenitor cell), (ii) genes determining the terminal differentiation of chondrocytes, and (iii) genes, the products of which trigger breakdown of the cartilaginous matrix.

Epidemiology of OA

OA, erroneously called degenerative joint disease, represents the failure of a diarthrodial (movable, synovial-lined) joint. In idiopathic (primary) OA, the most common form of the disease, no predisposing factor is apparent. Secondary OA is pathologically indistinguishable from idiopathic OA but is attributable to an underlying cause. OA is the most common of all human joint disorders and is the most prevalent arthritic condition in the United States and around the world. Estimates of OA prevalence based on clinical evaluation in various studies show that more than 90% of the population over the age of 70 has OA. The invention is aimed at novel avenues of therapy and prevention of the disease.

Pathogenesis of OA

OA is a heterogeneous group of conditions that lead to joint symptoms and signs associated with defective integrity of articular cartilage, in addition to related changes in the underlying bone at the joint margins. OA may be either idiopathic (i.e., primary) or secondary to other medical conditions (inflammatory, biochemical, endocrine-related, metabolic, and anatomic or developmental abnormalities). Age is the most powerful risk factor for OA but major trauma and repetitive joint use are also important risk factors for OA. The pattern of joint involvement in OA is also influenced by prior vocational or avocational overload.

The disease has two general stages: (1) compensated and (2) decompensated. Currently, most investigators feel that the primary changes occur in cartilage extracellular matrix due to exogenous reasons (i.e., load, injury, etc.). Then, a defect in the collagen network of the cartilage is apparent, and lysosomal enzymes and secreted proteases (MMPs, plasmin, cathepsins) probably account for the observed initial alterations in cartilage matrix. Their synthesis and secretion are stimulated by IL-1 or by other factors (e.g., mechanical stimuli). In the initial stage of the disease, compensatory cellular response is activated. Secreted by chondrocytes, protease inhibitors like TIMP and PAI-1 work to stabilize the system by opposing the protease activity. Growth factors such as IGF-1 and TGF-β are implicated in repair processes that may heal the lesion or at least stabilize the process by activating proliferation of cells of chondrogenic lineage. Finally, this leads to the accumulation of hypertrophic chondrocytes. The latter cells have marked biosynthetic activity that is expressed in increasing the proteoglycan (PG) concentration, associated with thickening of the cartilage (“compensated” OA). The compensatory mechanisms may maintain the joint in a reasonably functional state for years. However, the repair tissue does not hold up and the rate of PG synthesis falls off with full-thickness loss of cartilage. This marks the decompensated stage of OA. Following the destruction of the articular cartilage, there is migration of progenitor cells to the site of tissue damage. These cells proliferate and differentiate into four cell types: osteoblasts, chondroblasts, chondroclasts and fibroblasts, which combine to form bony structures called osteophytes that protrude into the joint space, thus inhibiting its movement. Finally, gradual replacement of cartilage with bone occurs.

The reason for this phenomenon is unknown. One possibility is that in OA, the normal inhibitory growth control of articular chondrocytes or synovial membrane fibroblasts is altered. This enables accumulation of two types of cells that cannot be found in normal articular cartilage: (1) immature mesenchymal and bone marrow cells with modified properties, and (2) hypertrophic articular chondrocytes. Previous results have clearly shown that hypertrophic chondrocytes may trigger osteogenesis by secretion of angiogenic and osteogenic factors (Homer, A., Bishop, N. J., Bord S., Beeton, C., Kelsall, A. W., Coleman, N. and Compston, J. E. (1999). Immunolocalisation of vascular endothelial growth factor (VEGF) in human neonatal growth plate cartilage. J. Anat. 194: 519-524).

In OA, therapeutic interference may target three main processes:
a) inhibition of initial cartilage damage—one of the accepted therapeutic strategies, combining recommendations to reduce the physical pressure on the joint and treatment with inhibitors of metalloproteinases;
b) inhibition or attenuation of total cartilage destruction at later stages—implies the therapeutic activation of processes connected to cartilage rehabilitation, namely, the promotion of proper differentiation of mesenchymal progenitors into mature chondrocytes capable of producing fully functional articular cartilage tissue;
c) inhibition or attenuation of osteophyte formation at the end stage of the disease —implies the therapeutic inhibition of ectopic osteogenesis at the site of articular cartilage, based on the fact that OA is characterized not only by degeneration of cartilage but also by ectopic osteogenesis.

Therefore, the inventors set out to identify target genes that code for specific factors that stimulate or inhibit the differentiation of progenitor cells to chondrocytes and/or stimulate or inhibit the differentiation of progenitor cells to osteoblasts.

The inventors employed both an in vitro cell system (Human Mesenchymal Stem Cells) and an ex vivo organ culture (fetal epiphyses grown in a joint simulator) to discover genes that may be important in the development of the disease. The Human Mesenchymal Stem Cells (HMSC) were used to conduct a series of gene expression profiling experiments. These cells were subjected to various treatments that mimic cartilage rehabilitation (IGF-1) or OA initiation and development (e.g., IL1-β, bFGF-2, mechanical stress). In addition to the in vitro studies, ex vivo experiments were also employed, as they better reflect genetic events occurring in the context of complex tissue containing different interacting types of cells. The gene expression profiles corresponding to various applied treatments were studied by microarray hybridization and analyzed by applicant's bioinformatics tools. The gene expression patterns obtained indicated that the chosen in vitro cell system accurately reflects the processes that occur in the OA joint in vivo since many genes known to be markers of OA were identified by the inventors as displaying the expected type of behavior.

Entire Isolated Epiphyses Grown in Joint Simulator

In addition to the in vitro experiments, the inventors have also used an ex vivo model of isolated fetal human epiphyses grown in a joint simulator, described below. This special device was developed by Cohen, I., Robinson, D., Cohen, N., Nevo, Z. (2000) Storing live embryonic and adult human cartilage grafts for transplantation using a joint simulating device. Biomaterials, 21:2117-2123, who showed that growing isolated epiphyses in this simulator causes marked proliferation of human mesenchymal stem cells, their differentiation to mature functional chondrocytes and a remarkable synthesis of high quality matrix. In contrast, epiphyses kept in regular tissue culture conditions show massive apoptosis and necrosis of the joint tissue. This model is advantageous since it reflects genetic events that take place in the context of complex tissue.

The joint simulator consists of a sterile growth chamber, fed by a closed tube system from a larger medium reservoir. Thus the cartilage positioned in the joint simulator is constantly irrigated by fresh medium enriched with CO₂. Normal articular cartilage in vivo is fed by synovial fluid pumped by hydrostatic forces generated by joint movements and loading. The deeper layer of cartilage, particularly of growing layers prior to the calcification of the tide mark, is fed by blood vessels. Thus, two patterns of pulsation are present in normal articular cartilage, i.e., that of joint motion and that of blood circulation. In the joint simulator, tissue is exposed to a continuous flow provided at the optimal rate (defined previously) of 570 ml/hour. The peristaltic pump generates pressure of a sinusoid pattern similar in range to systolic blood pressure (150 mm Hg, 100 pulses/min). In summary, the advantages of the joint simulator are:
ongoing perfusion, ensuring an abundant supply of nutrients and avoiding accumulation of waste products, and
stimulation of cartilage growth by mimicking hydrostatic and gravitational forces that act upon the articular cartilage.

Changes in gene expression caused by IL1-β, FGF-2 and/or mechanical stress, which are known osteogenic factors, may be connected to OA development and, therefore, should be opposed by therapeutic intervention. Surprisingly, it has been found by the present inventors that one of the genes found to be up-regulated by IL1-β treatment is the ISLR gene. This implied that the ISLR gene might be involved in the OA pathway. ISLR was thus selected as a target for further development of drugs for the treatment of OA in mammals.

Thus, as a first aspect, the present invention relates to a method for the treatment of a subject in need of treatment for OA or for RA, and related diseases, this method comprising administering to said subject an amount of the ISLR gene, gene product, agonist, or antagonist thereof effective to thereby treat the subject.

ISLR has been shown to have high structural similarity to gene products implicated in the development of osteogenesis. Therefore, another aspect of the present invention relates to a method for the treatment of a subject in need of treatment for OP, this method comprising administering to said subject an amount of the ISLR gene, gene product, agonist, or antagonist thereof effective to thereby treat the subject.

### SUMMARY OF THE INVENTION

The present invention provides human IL1-β-induced genes, and genes involved in bone formation, in particular ISLR, and their functional equivalents, expression products of such genes, uses for such genes and expression products for treatment, prevention and control of osteoarthritis (OA) and cartilage damage associated therewith, and of rheumatoid arthritis (RA) and of osteopenia (low bone density) that includes osteoporosis and periodontosis and treatment of symptoms thereof, and a method for promotion of bone building and bone repair that includes bone fracture healing and bone elongation. The invention further provides diagnostic, treatment, prevention and control methods or processes as well as compositions.

### BRIEF DESCRIPTION OF THE FIGURES

FIG. 1 presents a structural diagram of (i) human 608 polypeptide (OCP gene), (ii) human Adlican and human Adlican-2 polypeptides, and (iii) human ISLR polypeptide, showing the major domains of the polypeptides. All four polypeptides comprise a string of leucine-rich regions (LRR) near the N-terminus (with the exception of human Adlican-2), covering about 200-250 amino acids, plus a string of 10 Ig domains in the C-terminus half of the molecule (with the exception of ISLR, which is truncated at the C-terminus when compared to the other three polypeptides).

FIG. 2 presents the amino acid sequence alignment of (i) human Adlican (SEQ ID NO:3), (ii) human Adlican-2 full amino acid predicted sequence, as determined by the inventors (SEQ ID NO:4), and (iii) human ISLR (SEQ ID NO:2).

FIG. 3(a) presents the polynucleotide coding sequence of human ISLR (SEQ ID NO:1).

FIG. 3(b) presents the derived amino acid sequence of human ISLR (SEQ ID NO:2

FIG. 4. Expression of the ISLR gene in intact articular cartilage revealed by in situ hybridization.

(a, b)—Brightfield (a) and darkfield (b) images of intact cartilage section at low magnification showing little or no hybridization signal in chondrocytes.

(c, d)—Brightfield microphotographs at high magnification showing little (c) or no (d) expression in intact chondrocytes.

FIG. 5. Expression of ISLR gene in osteoarthritic (eroded) articular cartilage.

(a, b) Brightfield (a) and darkfield (b) microphotographs of articular cartilage section in erosion area showing strong hybridization signal in activated chondrocytes.

(c) Blowup of part of the section presented in (a) showing varying level of expression in activated chondrocytes.

The following Detailed Description, given by way of example, but not intended to limit the invention to specific embodiments described, may be understood in conjunction with the above Figures.

### DETAILED DESCRIPTION OF THE INVENTION

ISLR (Immunoglobulin Superfamily Containing Leucine-Rich Repeat).

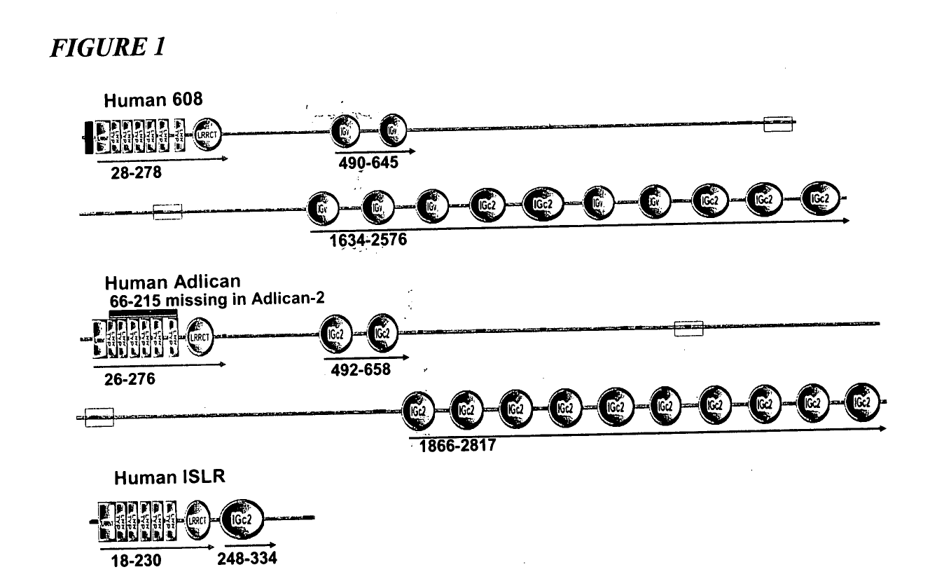

The present invention is related to ISLR, the function of which was not previously known, and the expression of which has herein been shown to be up-regulated by IL1-β on Human Mesenchymal Stem Cells (HMSCs). ISLR is structurally similar to OCP, Adlican and Adlican-2, at the N-terminus of the latter three molecules (see FIG. 1; see also PCT patent application, International Publication No. WO 99/60164, Publication date: 25 Nov. 1999, and PCT patent application, International Publication No WO 02/46364 PCT patent Publication date: 13 Jun. 2002, both assigned to one of the co-assignees of the instant application. The description therein of OCP demonstrates, without being bound by theory, how ISLR may function. Several functional features identify OCP as a specific early marker of osteo- or chondro-progenitor cells, as well as an inducer of osteoblast proliferation and differentiation.

These above-referenced PCT patent applications disclose the discovery of an isolated nucleic acid molecule, and the complement thereof, encoding the 608 protein (herein termed the “OCP” protein) or a functional portion thereof or a polypeptide, which is at least substantially homologous or identical thereto, as described in the above two PCT applications which are fully incorporated herein by reference. These applications discuss the full description and validation of the OCP gene, polypeptide encoded thereby, antibodies thereof, and a receptor thereof.

The instant invention provides for an isolated nucleic acid molecule encoding the ISLR protein, which displays high structural similarity to the OCP protein, the complement thereof, and a functional portion thereof.

The invention further encompasses an isolated polynucleotide encoding the ISLR polypeptide of nucleotide sequence as presented in SEQ ID NO:1, and a composition comprising said polynucleotide. By the term ISLR gene is meant a nucleotide acid sequence of SEQ ID NO:1 and homologs of the sequence having at least 95% homology, preferably at least 97% or most preferably at least 99% homology or nucleic acid sequences which bind to the ISLR gene under conditions of highly stringent hybridization, which are well-known in the art (for example Ausubel et al., Current Protocols in Molecular Biology, John Wiley and Sons, Baltimore, Md. (1988), updated in 1995 and 1998). Stringency conditions are a function of the temperature used in the hybridization experiment and washes, the molarity of the monovalent cations in the hybridization solution and in the wash solution(s) and the percentage of formamide in the hybridization solution The hybridization rate is maximized at a Ti (incubation temperature) of 20-25° C. below Tm for DNA:DNA hybrids and 10-15° C. below Tm for DNA:RNA hybrids. It is also maximized by an ionic strength of about 1.5M Na⁺. The rate is directly proportional to duplex length and inversely proportional to the degree of mismatching. The Tm of a perfect hybrid may be estimated for DNA:DNA hybrids using the equation of Meinkoth et al (1984)“Hybridization of nucleic acids immobilized on solid supports”, Anal Biochem 138:267-284.

By the term ISLR polypeptide is meant a polypeptide sequence of SEQ ID NO:2 and homologs of the sequence having at least 95% homology preferably at least 97% or most preferably at least 99% homology.

The ISLR gene and polypeptide are described in a Genebank entry dated Feb. 5, 1999, and in Nagasawa et al (1997) Genomics 44 (3):273-279 entitled Cloning of the cDNA for a new member of the immunoglobulin superfamily (ISLR) containing leucine-rich repeat (LRR) and Nagasawa et al (1999) Genomics 61(1):37-43. The ISLR gene/polypeptide is also described in PCT patent applications WO 00/077037, EP patent application publication no. EP 1130094, PCT patent application WO 01/040466, PCT patent application WO 02/000677 and PCT patent application WO 02/085285. PCT patent application WO 02/018608 and Tice et al, J Biol Chem (2002);277(16):14329-35) both relate to ISLR and Wnt signalling. Wnt signalling is also described in Tufan and Tuan (2001), The FASEB Journal, express article 10.1096/fj.00-0784fje.

None of the above publications provide any evidence of the relationship of the ISLR gene and polypeptide to osteoporosis and osteoarthritis.

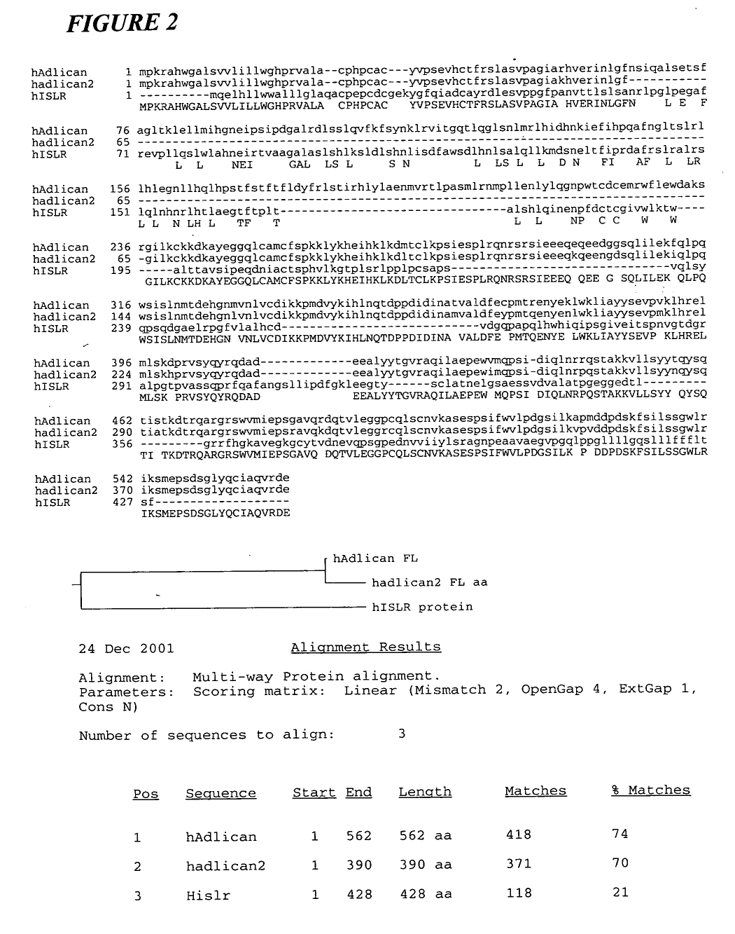

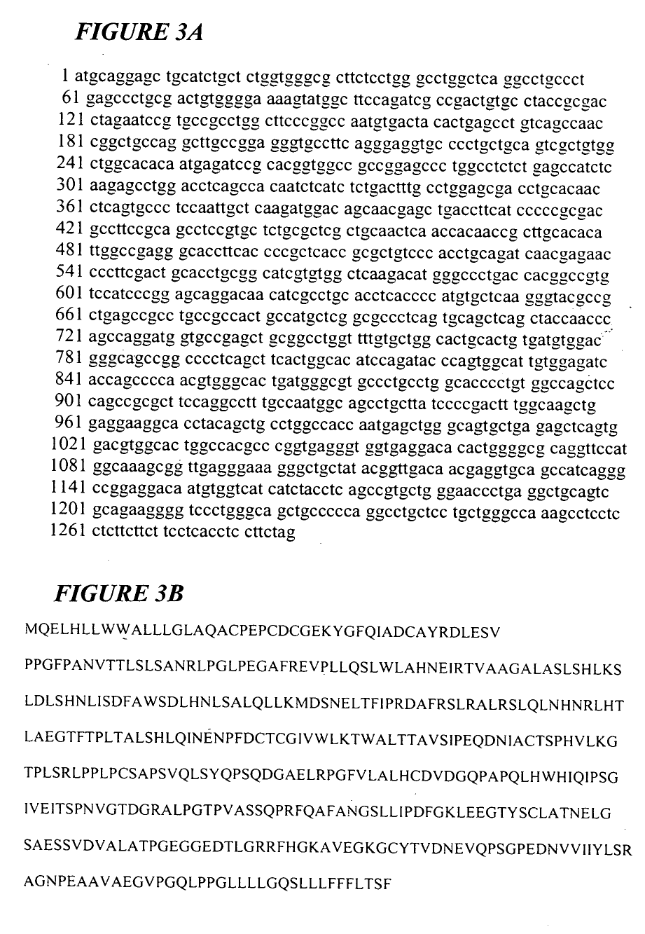

Particular fragments of the ISLR polypeptide include amino acids 1-50, 51-100, 101-150, 151-200, 201-250, 251-300, 301-350, 351400 and 401-428 of the sequence shown in FIG. 2. Further particular fragments of the ISLR polypeptide include amino acids 25-74, 75-124, 125-174, 175-224, 225-274, 275-324, 325-374 and 375-428 of the sequence shown in FIG. 2.

“RNA” as used herein refers to RNA isolated from cell cultures, cultured tissues or cells or tissues isolated from organisms which are differentiated, exposed to a chemical compound, infected with a pathogen, or otherwise stimulated. As used herein, “translation” is defined as the synthesis of protein encoded by an mRNA template.

As used herein, stimulation of translation, transcription, stability or transportation of unknown target mRNA or stimulating element, includes chemically, pathogenically, physically, or otherwise inducing or repressing an mRNA population encoded by genes derived from native tissues and/or cells under pathological and/or stress conditions. In other words, stimulating the expression of an mRNA with a stress-inducing element or “stressor” includes, but is not limited to, the application of an external cue, stimulus, or stimuli that stimulate(s) or initiate(s) translation of an mRNA stored as untranslated mRNA in the cells from the sample. The stressor may cause an increase in stability of certain mRNAs, or induce the transport of specific mRNAs from the nucleus to the cytoplasm. The stressor may also induce specific gene transcription. In addition to stimulating translation of mRNA from genes in native cells/tissues, stimulation can include induction and/or repression of genes under pathological and/or stress conditions. The method utilizes a stimulus or stressor to identify unknown target genes regulated at the various possible levels by the stress-inducing element or stressor.

More in particular, with respect to the polynucleotide representing the ISLR gene and polypeptide expressed from it, the invention further comprehends isolated and/or purified polynucleotides and isolated and/or purified polypeptides having at least about 70%, preferably at least about 75% or about 77% homology, advantageously at least about 80% or about 83%, more advantageously at least about 85% or about 87% homology, even more advantageously, at least about 90% or about 93% homology, and most advantageously at least about 95%, e.g., at least about 97%, about 98%, about 99% or even about 100% homology thereto. The invention also comprehends that these nucleic acid molecules and polypeptides can be used in the same fashion as the herein or aforementioned nucleic acid molecules and polypeptides.

Nucleotide sequence homology can be determined using the “Align” program of Myers and Miller, ((1988) CABIOS 4:11-17) and available at NCBI. Alternatively or additionally, the term “homology”, for instance, with respect to a nucleotide or amino acid sequence, can indicate a quantitative measure of homology between two sequences. The percent sequence homology can be calculated as (N_{ref}−N_{dif})*100/N_{ref}, wherein N_{dif} is the total number of non-identical residues in the two sequences when aligned and wherein N_{ref} is the number of residues in one of the sequences. Hence, AGTCAGTC has a sequence similarity of 75% to AATCAATC (N_{ref}=8; N_{dif}=2).

Alternatively or additionally “homology”, with respect to sequences, can refer to the number of positions with identical nucleotides or amino acid residues divided by the number of nucleotides or amino acid residues in the shorter of the two sequences wherein alignment of the two sequences can be determined in accordance with the Wilbur and Lipman algorithm ((1983) Proc. Natl. Acad. Sci. USA 80:726), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc., CA). When RNA sequences are said to be similar, or to have a degree of sequence identity or homology with DNA sequences, thymidine (T) in the DNA sequence is considered equal to uracil (U) in the RNA sequence (see also alignment used in the Figures). RNA sequences within the scope of the invention can be derived from DNA sequences or their complements, by substituting thymidine (T) in the DNA sequence with uracil (U).

Additionally or alternatively, amino acid sequence similarity or identity or homology can be determined, for instance, using the BlastP program (Altschul et al., Nucl. Acids Res. 25:3389-3402) and available at NCBI. The following references provide algorithms for comparing the relative identity or homology of amino acid residues of two proteins, and additionally or alternatively, with respect to the foregoing, the teachings in these references can be used for determining percent homology or identity (Smith et al. (1981) Adv. Appl. Math. 2:482-489; Smith et al. (1983) Nucl. Acids Res. 11:2205-2220; Devereux et al. (1984) Nucl. Acids Res. 12:387-395; Feng et al. (1987) J. Molec. Evol. 25:351-360; Higgins et al. (1989) CABIOS 5:151-153; and Thompson et al. (1994) Nucl. Acids Res. 22:4673-4680).

The genes disclosed herein and expression products, as well as genes identified by the herein disclosed methods and expression products thereof and the compositions comprising Adlican, Adlican-2, and ISLR or the Adlican, Adlican-2, and ISLR gene (including “functional” variations of such expression products, and truncated portions of herein defined genes such as portions of herein defined genes which encode a functional portion of an expression product), are useful in treating, preventing, controlling or diagnosing mechanical stress conditions or absence of reduced mechanical stress conditions.

As described herein ISLR, including functional portions thereof, may be used in all methods suitable for OCP, Adlican and Adlican-2. The structural homology between ISLR and Adlican, Adlican-2, and human OCP provides this novel use of the ISLR protein. Adlican is provided, for instance, in AF245505.1:1.8487. Adlican is named for “ADhesion protein with Leucine-rich repeats has Immunoglobulin domains related to perleCAN”; and shows elevated expression in cartilage from OA patients. The Adlican, Adlican-2, and ISLR gene, or functional portions thereof may likewise be used for any purpose described previously for an OCP gene. The invention further encompasses compositions comprising a physiologically acceptable excipient and at least one of Adlican, Adlican-2, and ISLR, the Adlican, Adlican-2, and ISLR gene and antibodies specific to Adlican, Adlican-2, and ISLR.

Since OCP expression is detected mostly in early committed osteo-chondroprogenitors and is associated with their proliferation, reduction of OCP level in blood may be indicative for states like OP which are accompanied by reduction in the number of early committed osteo-chondroprogenitors.

The medicament or treatment may be any conventional medicament or treatment for OP. Alternatively, or additionally, the medicament or treatment may be the particular protein of the gene detected in the inventive methods, or that which inhibits said protein, e.g., binds to it. Similarly, additionally, or alternatively, the medicament or treatment may be a vector comprising the isolated polynucleotide of the sequence as presented in SEQ ID NO:1 that expresses the protein of the gene detected in the inventive methods or that which inhibits expression of that gene; again, for instance, that which can bind to it and/or otherwise prevent its transcription or translation. The selection of administering a protein or that which expresses it, or of administering that which inhibits the protein or the gene expression, can be performed without undue experimentation, e.g., based on down-regulation or up-regulation as determined by inventive methods (e.g., in the OP model). The invention further recites a composition comprising said vector.

The invention encompasses a method for preventing, treating or controlling osteopenia (low bone density) that includes osteoporosis and periodontosis and treatment of symptoms thereof and a method for promotion of bone building and bone repair that includes bone fracture healing and bone elongation, comprising administering an isolated polynucleotide of sequence as presented in SEQ ID NO:1 or functional portion thereof or a polypeptide comprising an expression product of said isolated polynucleotide or functional portion of the polypeptide or an antibody to the polypeptide or a modulator (e.g. an agonist or an antagonist) thereof, and accordingly, the invention comprehends uses of a polypeptide in preparing a medicament or therapy for such prevention, treatment or control.

The invention further recites a method for preparing a polypeptide comprising expressing the isolated nucleic acid molecule of sequence as presented in SEQ ID NO:1. The invention provides for a method for preparing a polypeptide comprising expressing the polynucleotide of the vector as presented above.

The invention further provides for a method for preventing, treating or controlling conditions connected to activation and proliferation of osteochondroprogenitors like e.g. OA, RA, osteopetrosis, osteosarcoma, chondrosarcoma, or osteosclerosis symptoms in a subject, comprising administering an isolated polynucleotide of nucleotide sequence as presented by SEQ ID NO:1 or functional portion thereof or a polypeptide comprising an expression product of the said isolated polynucleotide or functional portion of the polypeptide or an antibody to the polypeptide or a modulator e.g. an agonist or an antagonist thereof.

The invention provides for an isolated polypeptide, wherein the polypeptide is identified as the ISLR protein, or a functional portion of the ISLR protein, or a polypeptide which is at least substantially homologous or identical thereto. This polypeptide may either preserve the activity of full length ISLR or act as its competitive inhibitor depending on the indication.

The invention further provides for a method for preventing, treating or controlling OA, osteopetrosis, osteosarcoma, chondrosarcoma, or osteosclerosis symptoms in a subject, comprising administering the isolated ISLR polypeptide, a biologically active fragment thereof, or agonist or antagonist thereof, or a neutralizing monoclonal antibody thereof.

The invention further provides for a method of using the receptor of the ISLR polypeptide to identify other polypeptides that bind to, associate with or block the receptor, for determining binding constants and degree of binding of the polypeptides, and for testing the functioning of such polypeptides utilising the receptor, crystalline receptor preparations, or membrane receptor preparations. Further provided for is a polypeptide that binds to the receptor described above, or an agonist or an antagonist thereof.

Screening Assays

Many types of compounds may be tested to determine if they are modulators that affect expression of the gene (transcription or translation) or the activity of the gene product (polypeptide) of interest. Examples of such compounds are small chemical molecules, antibodies preferably neutralizing antibodies or fragments thereof including single chain antibodies, antisense oligonucleotides, antisense DNA or RNA molecules, proteins, polypeptides and peptides including peptido-mimetics and dominant negatives, and expression vectors; see discussion of modulators below.

Many types of screening assays are known to those of ordinary skill in the art. The specific assay that is chosen depends to a great extent on the activity of the candidate gene or the protein expressed thereby. Thus, if it is known that the expression product of a candidate gene has enzymatic activity, then an assay which is based on inhibition (or stimulation) of the enzymatic activity can be used. If the candidate protein is known to bind to a ligand or other interactor (interacting molecule), then the assay can be based on the inhibition of such binding or interaction. When the candidate gene is a known gene, then many of its properties can also be known, and these can be used to determine the best screening assay. If the candidate gene is novel, or its function is novel, then some analysis and/or experimentation is appropriate in order to determine the best assay to be used to find inhibitors of the activity of that candidate gene. The analysis can involve a sequence analysis to find domains in the sequence that shed light on its activity. Other experimentation described herein to identify the candidate gene and its activity can also be engaged in, so as to identify the type of screen that is appropriate in order to find modulators, i.e. inhibitors or stimulators (enhancers), as the case may be, for the candidate gene or the protein encoded thereby.

As is well known in the art, such screening assays can be cell-based or non-cell-based. The cell-based assay is performed using eukaryotic cells, and such cell-based systems are particularly relevant in order to directly measure the activity of candidate genes that are involved in proliferation or differentiation. One way of running such a cell-based assay uses tetracycline-inducible (Tet-inducible) gene expression. Tet-inducible gene expression is well known in the art (e.g., Hofmann et al., 1996; Proc Natl Acad Sci, 93(11):5185-5190).

Tet-inducible retroviruses have been designed to incorporate the Self-inactivating (SIN) feature of a 3′ LTR enhancer/promoter retroviral deletion mutant. Expression of this vector in cells is virtually undetectable in the presence of tetracycline or other active analogs. However, in the absence of Tet, expression is turned on within a maximum of 48 hours after induction, with uniform increased expression of the entire population of cells that harbor the inducible retrovirus, thus indicating that expression is regulated uniformly within the infected cell population.

When dealing with candidate genes having a particular function, Tet-inducible expression increases the expression of that function in target cells. One can screen for chemical compounds able to rescue the cells from the gene-triggered increase or decrease in the specified function.

If the gene product of the candidate gene phosphorylates a specific target polypeptide, a specific reporter gene construct can be designed such that phosphorylation of this reporter gene product causes its activation, which can be followed by a color reaction. The candidate gene can be specifically induced, using the Tet-inducible system discussed above, and a comparison of induced versus non-induced genes provides a measure of reporter gene activation.

In a similar indirect assay, a reporter system can be designed that responds to changes in protein-protein interaction of the candidate polypeptide. If the reporter responds to actual interaction with the candidate protein, a color reaction occurs.

One can also measure inhibition or stimulation of reporter gene activity by modulation of its expression levels via the specific candidate promoter or other regulatory elements. A specific promoter or regulatory element controlling the activity of a candidate gene is defined by methods well known in the art. A reporter gene is constructed that is controlled by the specific candidate gene promoter or regulatory elements. The DNA containing the specific promoter or regulatory agent is actually linked to the gene encoding the reporter. Reporter activity depends upon specific activation of the promoter or regulatory element. Thus, inhibition or stimulation of the reporter gene is a direct assay of inhibition or stimulation of the candidate gene, respectively (e.g., Komarov et al., 1999, Science, 285,1733-1737; Storz et al., 1999, Analytical Biochemistry, 276, 97-104).

Design of various non-cell-based screening assays are also well within the skill of those of ordinary skill in the art. For example, if enzymatic activity is to be measured, such as if the candidate protein has a kinase activity, the target protein can be defined and specific phosphorylation of the target can be followed. The assay can involve either inhibition of target phosphorylation or stimulation of target phosphorylation, both types of assay being well known in the art, (e.g., Mohney et al., 1998 J.Neuroscience, 18, 5285; Tang et al., (1997) J. Clin. Invest. 100, 1180) for measurement of kinase activity.

One can also measure in vitro interaction of a candidate polypeptide with interactors. In this screen, the candidate polypeptide is immobilized on beads. An interactor, such as a receptor ligand, is radioactively labeled and added. When it binds to the candidate polypeptide on the bead, the amount of radioactivity carried on the beads (due to interaction with the candidate polypeptide) can be measured. The assay indicates inhibition of the interaction by measuring the amount of radioactivity on the bead. Any of the screening assays according to the present invention can include a step of identifying the chemical compound (as described above) which tests positive in the assay, and can also include the further step of producing as a medicament that which has been so identified. It can also include steps of improving the chemical compound to increase its desired activity before incorporating the improved chemical compound into a medicament. It is considered that medicaments comprising such compounds are part of the present invention. The use of any such compounds identified for modulation ( inhibition or stimulation) of the specified function of the gene product is also considered to be part of the present invention. Modulation of the expression of an ISLR gene, or of ISLR polypeptide activity, may be useful for accomplishing one or more of the following: growth, proliferation, differentiation and apoptosis of chondrocyte cells, bone progenitor cells and mesenchymal cells, which would be useful for treatment of osteoarthritis and other bone-related diseases including rheumatoid arthritis and also osteopetrosis, osteosarcoma, chondrosarcoma, or osteosclerosis. Measurement of the expression of an ISLR gene, or of ISLR polypeptide activity, can be achieved directly by methods known in the art and as herein referenced, and additionally one or more of the following can also be used as an end point indication in an evaluating method growth, proliferation, differentiation and apoptosis of chondrocyte cells, bone progenitor cells, mesenchymal cells. An end point indication can also be development of arthritis.

Thus this application is directed to a process for identifying a chemical compound that modulates expression of an ISLR gene which comprises:
(a) contacting a cell expressing the ISLR gene with the compound; and
(b) determining the ability of the compound to modulate expression of the ISLR gene as compared to a control.

In a preferred embodiment of the above process the cell in the contacting step (a) has been transfected or transduced by the ISLR gene and wherein the expression of the ISLR gene is associated with osteoarthritis or rheumatoid arthritis or OP or other bone diseases or conditions. Furthermore this application is directed to a process of preparing a pharmaceutical composition which comprises:
(a) determining whether a chemical compound modulates expression of an ISLR gene by using the above screening; and
(b) admixing said compound with a pharmaceutically acceptable carrier.

Also, this application is directed to a process of screening a plurality of chemical compounds not known to modulate expression of an ISLR gene to identify a compound which stimulates or inhibits expression of an ISLR gene which comprises:
(a) contacting a cell expressing the ISLR gene with the plurality of chemical compounds not known to modulate expression of an ISLR gene, under conditions permitting expression of the gene;
(b) determining whether expression of an ISLR gene is modulated in the presence of one or more of the compounds present in the plurality, as compared to a control; and if so
(c) separately determining which compound or compounds present in the plurality modulate expression of an ISLR gene, so as to thereby identify the compound which modulates the expression of the gene. Additionally, the cell in said contacting step (a) may have been transfected or transduced by the ISLR gene.

Furthermore this application is directed to a process of identifying a modulator of ISLR gene expression or a modulator of ISLR polypeptide activity, whereby the identification is performed by the steps of:
a. obtaining a candidate modulator;
b. evaluating the effect of said candidate modulator as compared to a control on expression of an ISLR gene or activity of ISLR polypeptide by an evaluating method

Additionally, in the above process the evaluating method may comprise the steps of:
i. providing a test system comprising DNA encoding ISLR;
ii. contacting said system with the said test candidate ISLR modulator under conditions which normally lead to expression of ISLR; and
iii determining the effect of the test candidate modulator on an end-point indication as compared to a control.

Additionally, in various preferred embodiments of the above process the test system is an in vitro transfected cell culture comprising an exogenously expressed ISLR polypeptide, the cell culture is a chondrocyte cell culture, the test system is an ex vivo bone culture comprising an endogenously expressed ISLR polypeptide, the bone culture is an embryonic bone, the test system is an in vivo test system comprising an animal model, the end point indication is development of arthritis the development of arthritis is determined by paw thickness of said animal, wherein less increase of the size of the paw as compared to a control is indicative of inhibition of development of arthritis by said test candidate inhibitor, the animal model is a transgenic animal, the in vivo test system is an arthritic mammalian model, preferably an arthritic rat expressing endogenous ISLR, and the end point indication is development of arthritis.

Furthermore this application is directed to an additional process for identifying a chemical compound that modulates activity of an ISLR polypeptide which comprises:
(a) contacting a cell expressing the ISLR polypeptide with the compound; and
(b) determining the ability of the compound to modulate activity of an ISLR polypeptide as compared to a control.

A preferred embodiment of the above process is wherein said cell in said contacting step (a) has been transfected or transduced by the ISLR gene and wherein the expression of the ISLR gene is associated with osteoarthritis or rheumatoid arthritis or OP or other bone diseases or conditions.

This application is also directed to a process of preparing a pharmaceutical composition which comprises:
(a) determining whether a chemical compound modulates activity of an ISLR polypeptide by using one or more of the above methods; and
(b) admixing said compound with a pharmaceutically acceptable carrier.

This application is further directed to a process of preparing a pharmaceutical composition which comprises:
of screening a plurality of chemical compounds not known to modulate activity of an ISLR polypeptide to identify a compound which stimulates or inhibits expression of an ISLR polypeptide which comprises:
(a) contacting a cell expressing the ISLR gene with the plurality of chemical compounds not known to modulate activity of an ISLR polypeptide, under conditions permitting expression of the gene;
(b) determining whether activity of an ISLR polypeptide is modulated in the presence of one or more of the compounds present in the plurality, as compared to a control; and if so
(c) separately determining which compound or compounds present in the plurality modulate ISLR polypeptide activity, so as to thereby identify the compound which modulates the polypeptide activity.

In a preferred embodiment of the above process the cell in the contacting step (a) has been transfected or transduced by the ISLR gene and the expression of the ISLR gene is associated with osteoarthritis or rheumatoid arthritis or OP or other bone diseases or conditions.

This application is further directed to a process of identifying a modulator of ISLR polypeptide activity, whereby the identification is performed by the steps of:
a. obtaining a candidate ISLR polypeptide modulator;
b. evaluating the effect of said candidate modulator as compared to a control on ISLR polypeptide activity by an evaluating method.

In a preferred embodiment of the above process the evaluating method comprises the steps of:
i. providing a test system comprising DNA encoding ISLR;
ii. contacting said system with the said test candidate ISLR modulator under conditions which normally lead to expression of ISLR; and
iii determining the effect of the test candidate modulator on an end-point indication as compared to a control.

This application is further directed to a non cell-based process for identifying a compound which modulates ISLR polypeptide activity which comprises:
(a) measuring the binding of ISLR polypeptide to an interactor with which ISLR polypeptide interacts specifically in vivo;
(b) contacting ISLR polypeptide with said compound; and
(c) determining whether the activity of the ISLR polypeptide is affected by said compound.

This application is further directed to a process of preparing a pharmaceutical composition which comprises:
(a) determining whether a chemical compound modulates ISLR gene expression or ISLR polypeptide activity by using one or more of the above methods; and
(b) admixing said compound with a pharmaceutically acceptable carrier.

This application is further directed to kit for identifying a compound which modulates ISLR polypeptide activity comprising;
(a) ISLR polypeptide;
(b) a interactor with which ISLR interacts specifically in vivo; and
(c) means for measuring the interaction of ISLR polypeptide to the interactor.

In the practice of the invention, one can employ general methods in molecular biology. Standard molecular biology techniques known in the art and not specifically described are generally followed as in Sambrook et al. (1989, 1992) Molecular Cloning: A Laboratory Manual, Cold Spring Harbor Laboratory, New York; and Ausubel et al. (1989) Current Protocols in Molecular Biology, John Wiley and Sons, Baltimore, Md.

PCR comprising the methods of the invention is performed in a reaction mixture comprising an amount, typically between <10 ng-200 ng template nucleic acid; 50-100 pmoles each oligonucleotide primer; 1-1.25 mM each deoxynucleotide triphosphate; a buffer solution appropriate for the polymerase used to catalyze the amplification reaction; and 0.5-2 Units of a polymerase, most preferably a thermostable polymerase (e.g., Taq polymerase or Tth polymerase).

Antibodies:

Antibodies may be used in various aspects of the invention, e.g., in detection or treatment or prevention methods. By the term “antibodies” is included monoclonal antibodies (Mabs), polyclonal antibodies and also antibody fragments as described below. The antibodies are preferably recombinant and human or humanized, as described below, and are preferably neutralizing antibodies. Conveniently, antibodies may be prepared against the immunogen or antigenic portion thereof, for example, a synthetic peptide based on the sequence, or prepared recombinantly by cloning techniques or the natural gene product and/or portions thereof may be isolated and used as the immunogen. The genes are identified as set forth in the present invention and the gene product identified. Immunogens can be used to produce antibodies by standard antibody production technology well-known to those skilled in the art, as described generally in Harlow and Lane (1988) Antibodies: A Laboratory Manual, Cold Spring Harbor Laboratory, Cold Spring Harbor, N.Y.; and Borrebaeck (1992) Antibody Engineering-A Practical Guide, W.H. Freeman and Co. Antibody fragments having antibody functional activity can also be prepared from the antibodies and include Fab, F(ab′)₂, Fv and scFv prepared by methods known to those skilled in the art (Bird et al. (1988) Science 242:423-426). Any peptide having sufficient flexibility and length can be used as an scFv linker. Usually the linker is selected to have little to no immunogenicity. An example of a linking peptide is (GGGGS)₃, which bridges approximately 3.5 nm between the C-terminus of one V region and the N-terminus of another V region. Other linker sequences can also be used, and can provide additional functions, such as a means for attaching a drug or a solid support.

For producing polyclonal antibodies a host, such as a rabbit or goat, is immunized with the immunogen or an immunogenic fragment thereof, generally with an adjuvant and, if necessary, coupled to a carrier. Antibodies to the immunogen are then collected from the sera of the immunized animal. The sera can be adsorbed against related immunogens so that no cross-reactive antibodies remain in the sera, rendering the polyclonal antibody monospecific. Polyclonal antibodies specific to the entire ISLR putative protein are prepared by methods well known in the art, and as described herein (the structure of ISLR resembles the N-terminal portion of the OCP polypeptide). Polyclonal antibodies are identified and the recombinant active form of ISLR is prepared. The antibodies can be used for the identification of this protein e.g. in diagnostic assays.

For producing mAbs, an appropriate donor, generally a mouse, is hyperimmunized with the immunogen and splenic antibody-producing cells are isolated. These cells are fused to an immortal cell, such as a myeloma cell, to provide an immortal fused cell hybrid that secretes the antibody. The cells are then cultured in bulk and the mAbs are harvested from the culture media for use. Hybridoma cell lines provide a constant, inexpensive source of chemically identical antibodies and preparations of such antibodies can be easily standardized. Methods for producing mAbs are well known to those of ordinary skill in the art (see, e.g. U.S. Pat. No. 4,196,265).

For producing recombinant antibodies, mRNAs from antibody producing B-lymphocytes of animals, or hybridomas, are reverse-transcribed to obtain cDNAs (see, generally, Huston et al. (1991) Met. Enzymol. 203:46-88; Johnson and Bird (1991) Met. Enzymol. 203:88-99; and Mernaugh and Mernaugh (1995) in Molecular Methods in Plant Pathology (Singh and Singh eds.) CRC Press Inc., Boca Raton, Fla.:359-365). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

Antibodies can be bound to a solid support substrate or conjugated with a detectable moiety or be both bound and conjugated, as is well known in the art. For a general discussion of conjugation of fluorescent or enzymatic moieties see, Johnston and Thorpe (1982) Immunochemistry in Practice, Blackwell Scientific Publications, Oxford. The binding of antibodies to a solid support substrate is also well known in the art (for a general discussion, see Harlow and Lane (1988),supra; and Borrebaeck (1992) supra). The detectable moieties contemplated with the present invention include, but are not limited to, fluorescent, metallic, enzymatic and radioactive markers such as biotin, gold, ferritin, alkaline phosphatase (ALP), galactosidase, peroxidase, urease, fluorescein, rhodamine, tritium, ¹³C and iodination.

Antibodies can also be used as an active agent in a therapeutic composition and such antibodies can be humanized, for instance, to enhance their effects (Huls et al. (1999) Nature Biotech. 17). “Humanized” antibodies are antibodies in which at least part of the sequence has been altered from its initial form to render it more like human immunoglobulins. In one version, the H chain and L chain C regions are replaced with human sequence. In another version, the CDR regions comprise amino acid sequences from the antibody of interest, while the V framework regions contain converted human sequences (see, for example, EP 0 329 400). In a third version, V regions are humanized by designing consensus sequences of human and mouse V regions, and converting residues outside the CDRs that are different between the consensus sequences. The invention encompasses humanized mAbs. The invention further encompasses human antibodies to these antigens.

The expression product of the ISLR gene or homologs or fragments (portions) thereof can be useful for generating antibodies such as monoclonal or polyclonal antibodies which are useful for diagnostic purposes or to block activity of expression products or portions thereof or of genes or a portion thereof, e.g., as therapeutics.

The genes of the present invention or portions thereof, e.g., a portion thereof which expresses a protein that functions the same as or analogously to the full length protein, or genes identified by the methods herein can be expressed recombinantly, e.g., in Escherichia coli or in another vector or plasmid for either in vivo expression or in vitro expression. The methods for making and/or administering a vector or a recombinant plasmid for expression of gene products of the invention or identified by the invention or a portion thereof either in vivo or in vitro can be any desired method, e.g., a method which is by or analogous to the methods disclosed in U.S. Pat. Nos. 4,603,112; 4,769,330; 5,174,993; 5,505,941; 5,338,683; 5,494,807; 4,394,448; 4,722,848; 4,745,051; 4,769,331; 5,591,639; 5,589,466; 4,945,050; 5,677,178; 5,591,439; 5,552,143; and 5,580,859; U.S. patent application Serial No. 920,197, filed Oct. 16, 1986; WO 94/16716; WO 96/39491; W091/11525; WO 98/33510; WO 90/01543; EP 0 370 573; EP 265785; Paoletti (1996) Proc. Natl. Acad. Sci. USA 93:11349-11353; Moss (1996) Proc. Natl. Acad. Sci. USA 93:11341-11348; Richardson (Ed) (1995) Methods in Molecular Biology 39, “Baculovirus Expression Protocols,” Humana Press Inc.; Smith et al. (1983) Mol. Cell. Biol. 3:2156-2165; Pennock et al. (1984) Mol. Cell. Biol. 4:399-406; Roizman, Proc. Natl. Acad. Sci. USA 93:11307-11312; Andreansky et al. Proc. Natl. Acad. Sci. USA 93:11313-11318; Robertson et al. Proc. Natl. Acad. Sci. USA 93:11334-11340; Frolov et al. Proc. Natl. Acad. Sci. USA 93:11371-11377; Kitson et al. (1991) J. Virol. 65:3068-3075; Grunhaus et al. (1992) Sem. Virol. 3:237-252; Ballay et al. (1993) EMBO J. 4:3861-3865; Graham (1990) Tibtech 8:85-87; Prevec et al. J. Gen. Virol. 70:429-434; Felgner et al. (1994) J. Biol. Chem. 269:2550-2561; (1993) Science 259:1745-1749; McClements et al. (1996) Proc. Natl. Acad. Sci. USA 93:11414-11420; Ju et al. (1998) Diabetologia 41:736-739; and Robinson et al. (1997) Sem. Immunol. 9:271-283.

The expression product generated by vectors or recombinants can also be isolated and/or purified from infected or transfected cells, e.g., to prepare compositions for administration to patients. However, in certain instances, it may be advantageous not to isolate and/or purify an expression product from a cell; for instance, when the cell or portions thereof enhance the effect of the polypeptide.

As used herein, the term “polypeptide” denotes, in addition to a polypeptide, a peptide and a full protein, as well as a fragment or fragments thereof.

As used herein, “treatment” refers to clinical intervention in an attempt to alter the natural course of the individual or cell being treated, and may be performed either for prophylaxis or during the course of clinical pathology. Desirable effects of the treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis.

An inventive vector or recombinant nucleotide expressing a gene or a portion thereof identified herein, or from a method herein, can be administered in any suitable amount to achieve expression at a suitable dosage level, e.g., a dosage level analogous to the herein mentioned dosage levels (wherein the gene product is directly present). The inventive vector or recombinant nucleotide can be administered to a patient or infected or transfected into cells in an amount of about at least 10³ pfu; more preferably about 10⁴ pfu to about 10¹⁰ pfu, e.g., about 10⁵ pfu to about 10⁹ pfu, for instance about 10⁶ pfu to about 10⁸ pfu. In plasmid compositions, the dosage should be a sufficient amount of plasmid to elicit a response analogous to compositions wherein the gene product or a portion thereof is directly present; or to have expression analogous to dosages in such compositions; or to have expression analogous to expression obtained in vivo by recombinant compositions. For instance, suitable quantities of plasmid DNA in plasmid compositions can be 1 g to 100 mg, preferably 0.1 mg to 10 mg, e.g., 500 g, but lower levels such as 0.1 mg to 2 mg or preferably 1 g-10 g may be employed.

Documents cited herein regarding DNA plasmid vectors can be consulted for the skilled artisan to ascertain other suitable dosages for DNA plasmid vector compositions of the invention, without undue experimentation.

Compositions for administering vectors can be as in or analogous to such compositions in documents cited herein or as in or analogous to compositions herein described, e.g., pharmaceutical or therapeutic compositions and the like.

Thus, the invention comprehends in vivo gene expression that is sometimes termed “gene therapy”. Gene therapy can refer to the transfer of genetic material (e.g., DNA or RNA) of interest into a host subject or patient to treat or prevent a genetic or acquired disease, condition or phenotype. The particular gene that is to be used or that has been identified as the target gene is identified as set forth herein. The genetic material of interest encodes a product (e.g., a protein, polypeptide, peptide or functional RNA), the production in vivo of which is desired. For example, the genetic material of interest can encode a hormone, receptor, enzyme, polypeptide or peptide of therapeutic value (for a review see, in general, the text “Gene Therapy”, Advances in Pharmacology, 40, Academic Press, 1997).

Two basic approaches to gene therapy have evolved: (1) ex vivo; and (2) in vivo gene therapy. In ex vivo gene therapy cells are removed from a patient and, while being cultured, are treated in vitro. Generally, a functional replacement gene is introduced into the cell via an appropriate gene delivery vehicle/method (e.g., transfection, homologous recombination) and an expression system as needed, and then the modified cells are expanded in culture and returned to the host/patient. These genetically altered and subsequently re-implanted cells have been shown to produce the transfected gene product in situ. In in vivo gene therapy, target cells are not removed from the subject; rather, the gene to be transferred is introduced into the cells of the recipient organism in situ, that is, within the recipient. Alternatively, if the host gene is defective, the gene is repaired in situ (Culver (1998) Antisense DNA & RNA Based Therapeutics, February, 1998, Coronado, Calif.). These genetically altered cells have been shown to produce the transfected gene product in situ.

The gene expression vehicle is capable of delivery/transfer of heterologous nucleic acid into a host cell. The expression vehicle may include elements to control targeting, expression and transcription of the nucleic acid in a cell-selective manner as is known in the art. It should be noted that often the 5′UTR and/or 3′UTR of the gene may be replaced by the 5′ UTR and/or 3′UTR of the expression vehicle. Therefore, as used herein, the expression vehicle may, as needed, not include the 5′UTR and/or 3′UTR shown in sequences herein and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any non-translated DNA sequence that works contiguously with the coding sequence (in cis) to change the basal transcription level dictated by the promoter. The expression vehicle can also include a selection gene as described herein.

Vectors can be introduced into cells or tissues by any one of a variety of known methods within the art. Such methods can be found generally described in Sambrook et al. (1989, 1992); Ausubel et al. (1989); Chang et al. (1995) Somatic Gene Therapy, CRC Press, Ann Arbor, Mich.; Vega et al. (1995) Gene Targeting, CRC Press, Ann Arbor, Mich.; Vectors: A Survey of Molecular Cloning Vectors and Their Uses, Butterworths, Boston Mass. (988); and Gilboa et al. (1986) BioTech. 4:504-512, as well as other documents cited herein and include, for example, stable or transient transfection, lipofection, electroporation and infection with recombinant viral vectors. In addition, see U.S. Pat. No. 4,866,042 for vectors involving the central nervous system; and also U.S. Pat. Nos. 5,464,764 and 5,487,992 for positive-negative selection methods.

Introduction of nucleic acids by infection offers advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed cell culture. Viral vectors can also be modified with specific receptors or ligands to alter target specificity through receptor-mediated events.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur. Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus, and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Delivery of gene products (products from herein defined genes, i.e., genes identified herein or by inventive methods or portions thereof) and/or antibodies or portions thereof and/or agonists or antagonists (collectively or individually “therapeutics”), and compositions comprising the same, as well as of compositions comprising a vector expressing gene products, can be performed without undue experimentation from this disclosure and the knowledge in the art.

The pharmaceutically “effective amount” for purposes herein is thus determined by such considerations as are known in the art. The amount must be effective to achieve improvement including, but not limited to, improved survival rate or more rapid recovery, or improvement or amelioration or elimination of symptoms and other indicators, e.g., of OP, for instance, improvement in bone density, as are selected as appropriate measures by those skilled in the art.

It is noted that humans are treated generally longer than are the mice or other experimental animals exemplified herein. Human treatment has a length proportional to the length of the disease process and drug effectiveness. The doses may be single doses or multiple doses over a period of several days, but single doses are preferred. Thus, one can scale up from animal experiments, e.g., rats, mice, and the like, to humans, by techniques from this disclosure and the knowledge in the art, without undue experimentation.

The present invention also provides a composition of the isolated nucleic acid molecule (gene encoding ISLR), a vector comprising the isolated nucleic acid molecule, a composition containing said vector and a method for preventing, treating or controlling bone diseases including, but not limited to, OA, RA, osteopenia, osteoporosis (OP), periodontosis osteopetrosis, osteosclerosis, osteosarcoma, chondrosarcoma, and bone fractures or low bone density or or other conditions involving mechanical stress or a lack thereof in a subject, comprising administering the inventive composition, or the inventive vector, and a method for preparing a polypeptide comprising expressing the isolated nucleic acid molecule or comprising expressing the polypeptide from the vector.

The present invention further provides a method for preventing, treating or controlling OA, RA, osteopetrosis, osteosclerosis, osteosarcoma, chondrosarcoma or osteopenia, osteoporosis (OP), periodontosis, and bone fractures or low bone density or other factors causing or contributing to OP or symptoms thereof or other conditions involving mechanical stress or a lack thereof in a subject, comprising administering an isolated nucleic acid molecule or functional portion thereof or a polypeptide comprising an expression product of the gene or functional portion of the polypeptide or an antibody to the polypeptide or a functional portion of the antibody. The invention thus further comprehends uses of such genes (nucleic acid molecules), expression products, antibodies and portions thereof, in the preparation of a medicament or therapy for such control, prevention or treatment.

This application is directed to a process of preparing a therapeutic composition for the treatment of a subject in need of a treatment for osteoarthritis, which process comprises the steps of:
a. obtaining by one or more of the above processes an amount of modulator sufficient to effect a substantial modulation, and
b. admixing said modulator with a pharmaceutically acceptable carrier.

In a preferred embodiment of the above process, the modulator is an inhibitor.

This application is also directed to a method of treating, controlling or preventing osteoarthritis, rheumatoid arthritis, osteopetrosis, osteosarcoma, chondrosarcoma, or osteosclerosis, in a subject in need of such treatment comprising administering to a subject an effective amount of a modulator of ISLR gene expression or ISLR polypeptide activity sufficient to effect a substantial modulation of ISLR activity so as to thereby treat the subject. This application is also directed to the use of an modulator of ISLR gene expression or ISLR polypeptide activity in the treatment of a subject in need of treatment for osteoarthritis and other diseases preferably rheumatoid arthritis and also osteopetrosis, osteosarcoma, chondrosarcoma, or osteosclerosis in an amount sufficient to effect a substantial modulation of ISLR gene expression or ISLR polypeptide activity so as to thereby treat the subject. This application is also directed to use of an modulator of ISLR gene expression or ISLR polypeptide activity in the preparation of a pharmaceutical composition for the treatment of a subject in need of treatment for osteoarthritis. This application is also directed a therapeutic composition for the treatment of a subject in need of treatment for osteoarthritis comprising an amount of modulator of ISLR gene expression or ISLR polypeptide activity sufficient to effect a substantial modulation of ISLR expression or activity, and a pharmaceutically or veterinarily acceptable carrier.

The present invention provides an isolated polypeptide encoded by an isolated polynucleotide. In one embodiment of the invention, the polypeptide is identified as human ISLR or a functional portion thereof or a polypeptide which is at least substantially homologous or identical thereto. Preferably, the functional portion comprises an N-terminal polypeptide having a molecular weight of 10 kD to 100 kD. The present invention also provides a composition comprising one or more isolated polypeptides, an antibody specific for the polypeptide or a functional portion thereof, a composition comprising the antibody, and a method for treating or preventing OP or periodontosis or fracture healing or bone elongation, in a subject, comprising administering to the subject a N-terminal polypeptide having a molecular weight of between 10 kD and 100 kD.

The present invention also provides a composition comprising one or more isolated polypeptides, an antibody specific for the polypeptide or a functional portion thereof, a composition comprising the antibody, and a method for treating or preventing OA, RA, osteopetrosis, osteosarcoma, chondrosarcoma, periodontosis or osteosclerosis, in a subject, comprising administering to the subject a N-terminal polypeptide having a molecular weight of between 10 kD and 100 kD.

The present invention additionally provides for a method of treating or preventing OA, RA, osteopetrosis, osteosarcoma, chondrosarcoma, periodontosis or osteosclerosis, comprising administering to a subject an effective amount of a chemical compound (small molecule) or a neutralizing mAb that inhibits the activity of the ISLR polypeptide, or the functional fragment thereof.

The modulator of ISLR expression (transcription or translation) or polypeptide activity may be inter alia a small chemical molecule which generally has a molecular weight of less than 2000 daltons, more preferably less than 1000 daltons. Other modulators may be antibodies preferably neutralizing antibodies or fragments thereof including single chain antibodies, antisense oligonucleotides, antisense DNA or RNA molecules, proteins, polypeptides and peptides including peptido-mimetics and dominant negatives, and expression vectors. These modulators may act as follows: small molecules may affect expression and/or activity; antibodies-only activity; all kinds of antisense-only expression; dominant negative and peptidomimetics-only activity; expression vectors may be used inter alia for delivery of antisense or dominant-negative.

In an embodiment of the present invention, a small molecule is administered to a target cell, tissue, or organism, such that the small molecule permeates the cell membrane of said target cell, or of the cell in the target tissue or organism and effects an activation or inactivation of a specified polypeptide therein.

In a preferred embodiment of the present invention, the modulator is an agonist of the target protein. In a more preferred embodiment of the present invention, the modulator is an antagonist (inhibitor) of the target protein.

Approaches have recently been developed that utilize small molecules, which can bind directly to proteins and can be used to alter protein function (see review, B. R. Stockwell, (2000) Nature Reviews/Genetics, 1, 116-125). Low molecular weight organic compounds can permeate the plasma membrane of target cells relatively easily and, therefore, methods have been developed for their synthesis. These syntheses, in turn, have yielded libraries that contain ligands for many proteins. Recent developments have brought a greatly increased variety of creatively selected, novel, small organic molecules that will function as powerful tools for perturbing biological systems. Such small molecules can be used to activate or inactivate specific members of a protein family.

As used herein, the term “subject”, “patient” and “host” include, but are not limited to, human, bovine, pig, mouse, rat, goat, sheep and horse and other mammals.

Those skilled in the art will recognize that the components of the compositions should be selected to be chemically inert with respect to the gene product and optional adjuvant or additive. This will present no problem to those skilled in chemical and pharmaceutical principles, or problems can be readily avoided by reference to standard texts or by simple experiments (not involving undue experimentation), from this disclosure and the documents cited herein.

The present invention provides for receptors of the expression product of the human IL1-β-induced genes and their functional equivalents, in particular ISLR, and methods or processes for obtaining and using such receptors. The receptors of the present invention are those to which the ISLR gene and its functional equivalents bind or associate as determined by conventional assays, as well as in vivo. For example, binding of the ISLR polypeptides of the instant invention to receptors can be determined in vitro, using candidate receptor molecules that are associated with lipid membranes (Watson, J. et al., Development of FlashPlate® technology to measure (³⁵S) GTP gamma S binding to Chinese hamster ovary cell membranes expressing the cloned human 5-HT1B receptor, Journal of Biomolecular Screening. Summer, 1998; 3 (2) 101-105; Komesli-Sylviane et al., Chimeric extracellular domain of type II transforming growth factor (TGF)-beta receptor fused to the Fc region of human immunoglobulin as a TGF-β antagonist, European Journal of Biochemistry. June 1998; 254 (3) 505-513); see, generally, Darnell et al., Molecular Cell Biology, 644-646, Scientific American Books, New York (1986)). Scanning electron microscopy (“SEM”), X-ray crystallography and reactions using labeled polypeptides are examples of conventional means for determining whether polypeptides are bound or associated with a receptor molecule. For instance, X-ray crystallography can provide detailed structural information to determine whether and to what extent binding or association has occurred (see, e.g., U.S. Pat. No. 6,037,117; U.S. Pat. No. 6,128,582 and U.S. Pat. No. 6,153,579). Further, crystallography, including X-ray crystallography, provides three-dimensional structures that show whether a candidate polypeptide ligand can or would bind or associate with a target molecule, such as a receptor (e.g., WO 99/45379; U.S. Pat. Nos. 6,087,478 and 6,110,672). Such binding or association shows that the receptor molecule is the receptor for the candidate polypeptide.

With the disclosures in the present specification of the inventive genes, expression products and uses thereof, those skilled in the art can obtain by conventional methods the receptors for the inventive expression products. The conventional means for obtaining the receptors include raising monoclonal antibodies (mAbs) to candidate receptors, purifying the receptors from a tissue sample by use of an affinity column, treatment with a buffer, and collection of the eluate receptor molecules. Other means of isolating and purifying the receptors are conventional in the art, for instance isolation and purification by dialysis, salting out, and electrophoretic (e.g. SDS-PAGE) and chromatographic (e.g., ion-exchange and gel-filtration, in additional to affinity) techniques. Such methods can be found generally described in Stryer, Biochemistry, 44-50, W.H. Freeman & Co., New York (3d ed. 1988); Darnell et al., Molecular Cell Biology, 77-80 (1986); Alberts et al., Molecular Biology of the Cell, 167-172, 193 Garland Publishing, New York (2^{nd} ed., 1989).

Sequencing of the isolated receptor involves methods known in the art, for instance, directly sequencing a short N-terminal sequence of the receptor, constructing a nucleic acid probe, isolating the receptor gene, and determining the entire amino-acid sequence of the receptor from the nucleic acid sequence. Alternatively, the entire receptor protein can be sequenced directly. Automated Edman degradation is one conventional method used to sequence, partially or entirely, a receptor protein, facilitated by chemical or enzymatic cleavage. Automated sequenators, such as an ABI-494 Procise Sequencer (Applied Biosystems) can be used (see, generally, Stryer, Biochemistry, 50-58 (3^{rd} ed., 1988)).

The invention provides methods or processes for using such receptors in assays, for instance, for identifying proteins or polypeptides that bind to, associate with or block the inventive receptors, determining binding constants and degree of binding, and for testing the effects of such polypeptides, for instance, utilising membrane receptor preparations (Watson (1998); Komesli-Sylviane (1998)). For instance, FlashPlate® (Perkin-Elmer, Massachusetts, USA) technology can be used with the present invention to determine whether and to what degree candidate polypeptides bind to and are functional with respect to a receptor of the invention.

This application also relates to a process for preventing, treating or controlling OA, RA, osteoporosis, or for fracture healing, bone elongation or osteopenia, periodontosis, or low bone density or other conditions involving mechanical stress or a lack thereof in a subject, comprising administering to the subject an effective amount of a modulator of ISLR activity.

Diagnostics:

In diagnosis, the sample is taken from a bodily fluid or from a tissue, preferably bone tissue; the bodily fluid is selected from the group of fluid consisting of blood, lymph fluid, ascites, serous fluid, pleural effusion, sputum, cerebrospinal fluid, lacrimal fluid, synovial fluid, saliva, stool, sperm and urine. Measurement of level of the ISLR polypeptide is determined by a method selected from the group consisting of immunohistochemistry, western blotting, ELISA, antibody microarray hybridization and targeted molecular imaging. Such methods are well-known in the art, for example for immunohistochemistry: M. A. Hayat (2002) Microscopy, Immunohistochemistry and Antigen Retrieval Methods: For Light and Electron Microscopy, Kluwer Academic Publishers; Brown C (1998): “Antigen retrieval methods for immunohistochemistry”, Toxicol Pathol; 26(6): 830-1); for western blotting: Laemmeli UK(1970): “Cleavage of structural proteins during the assembly of the head of a bacteriophage T4”, Nature;227: 680-685; and Egger & Bienz(1994) “Protein (western) blotting”, Mol Biotechnol; 1(3): 289-305); for ELISA: Onorato et al.(1998) “immunohistochemical and ELISA assays for biomarkers of oxidative stress in aging and disease”, Ann NY Acad Sci 20; 854: 277-90); for antibody microarray hybridization :Huang(2001) “Detection of multiple proteins in an antibody-based protein microarray system, Immunol Methods 1; 255 (1-2): 1-13); and for targeted molecular imaging: Thomas (2001). Targeted Molecular Imaging in Oncology, Kim et al (Eds)., Springer Verlag, inter alia.

Measurement of level of ISLR polynucleotide is determined by a method selected from: RT-PCR analysis, in-situ hybridization, polynucleotide microarray and Northern blotting. Such methods are well-known in the art, for example for in-situ hybridization Andreeff & Pinkel (Editors) (1999), “Introduction to Fluorescence In Situ Hybridization: Principles and Clinical Applications”, John Wiley & Sons Inc.; and for Northern blotting Trayhurn (1996) “Northern blotting”, Proc Nutr Soc; 55(1B): 583-9 and Shifman & Stein(1995) “A reliable and sensitive method for non-radioactive Northern blot analysis of nerve growth factor mRNA from brain tissues”, Journal of Neuroscience Methods; 59: 205-208 inter alia.

Diagnostics involving measurement of the ISLR polypeptide or polynucleotide may also be developed in accordance with diagnostic methods known in the art for diagnosis of OA or OP or similar diseases or conditions; see for example Ratcliffe A. et al. (1996) “Biochemical markers in synovial fluid identify early osteoarthritis of the glenohumeral joint”: Clin. Orthop: Sept (330): 45-53;Lohmaner L. S. (1997) “What is the current status of biochemical markers in the diagnosis, prognosis and monitoring of osteoathritis?”: Baillieres Clin Rheumatol: 11(4): 711-726; DeGroot, J. et al. (2002) “Molecular markers for osteoarthritis: the road ahead”: Current Opinion in Rheumatology: 14(5): 585-589; Thonar E. (1999) “Molecular markers of metabolic changes in osteoarthritis”: Osteoarthritis and Cartilage: 7:338-339; Petersson I. F. (1998) “Changes in cartilage and bone metabolism identified by serum markers in early osteoarthritis of the knee joint”: British Journal of Rheumatology: 37:46-50; Adachi J. D. (1996) “The correlation of bone mineral density and biochemical markers to fracture risk”: Calcif Tissue Int: 59(Suppl 1):S16-S19; Delmas P. D. et al. (2000) “The use of biochemical markers of bone turnover in osteoporosis”: Osteoporos Int: 6:S2-S17; Ebeling P. R. et al. (2001) “Role of biochemical markers in the management of osteoporosis”: Best Practice & Research Clinical Rheumatology: 15(3):385-400;Brown J. P. et al. (Nov. 12, 2002) “2002 clinical practive guidelines for the diagnosis and management of osteoporosis in Canada”: CMAJ: 167:S1-S34; Garnero P. et al. (1996) “New developments in biochemical markers for osteoporosis”: Calcif Tissue Int; 59(Suppl 1):S2-S9; and Arnaud C.D. (April 1996) “Osteoporosis: using ‘Bone markers’ for diagnosis and monitoring”: Geriatrics: 51(4):24-30, inter alia.

Thus this application is directed to a method for diagnosing osteoarthritis in a subject comprising determining, in a sample from the subject, the level of ISLR polypeptide, wherein a higher level of the polypeptide compared to the level of the polypeptide in a subject free of osteoarthritis is indicative of osteoarthritis, and wherein the polypeptide is selected from the group consisting of:
a. the ISLR polypeptide; and
b. polypeptides which are at least 70% homologous to the polypeptide of (a).

This application is also directed to a method for diagnosing osteoarthritis in a subject comprising determining, in a sample from the subject, the level of at least one polypeptide-encoding polynucleotide, wherein a higher level of the polynucleotide compared to the level of the polynucleotide in a subject free of osteoarthritis is indicative of osteoarthritis, and wherein the polynucleotide is selected from the group consisting of:
a. the polynucleotide encoding ISLR polypeptide
b. polynucleotides having sequences that differ from the polynucleotide in (a), without changing the polypeptide encoded thereby; and
c. polynucleotides which are at least 70% homologous to the polynucleotide of (a).

This application is also directed to a method for measuring the responsiveness of a subject to osteoarthritis treatment comprising determining the level of at least one polypeptide in a sample taken from the subject before treatment, and comparing it with the level of said polypeptide in a sample taken from the subject after treatment, a decrease in said level indicating responsiveness of said subject to the osteoarthritis treatment, wherein the polypeptide is selected from the group consisting of:
a. ISLR polypeptide; and
b. polypeptides which are at least 70% homologous to the polypeptide of (a).

This application is also directed to a method for measuring the responsiveness of a subject to a treatment for osteoarthritis comprising determining the level of at least one polypeptide-encoding polynucleotide in a sample taken from the subject before treatment, and comparing it with the level of said polynucleotide in a sample taken from the subject after treatment, a decrease in said level indicating responsiveness of said subject to the treatment for osteoarthritis, wherein the polynucleotide is selected from the group consisting of:
a. the ISLR polynucleotide;
b. polynucleotides having sequences that differ from the polynucleotide in (a), without changing the polypeptide encoded thereby; and
c. polynucleotides which are at least 70% homologous to the polynucleotide of (a).

These and other embodiments are disclosed or are obvious from and encompassed by the above “Detailed Description”.

A better understanding of the present invention and of its many advantages will be obtained from the following Examples, given by way of illustration and as a further description of the invention.

### EXAMPLE 1

Experimental Models for Identification of Genes Involved in the Development of OA and Cartilage Rehabilitation

Experimental Design of Model Systems

In order to understand the molecular mechanisms that accompany the pathogenesis of OA, the inventors used a panel of in vitro and ex vivo models. The compilation of the results derived from several complementary models facilitated the classification of related but distinct physiological conditions according to their gene expression patterns and, subsequently, selection of genes that are critical for a specific physiological or pathological process.

The experimental strategy undertaken was based on the following pre-existing knowledge:
1. Normal adult articular cartilage progenitor cells (HMSCs) kept in tissue culture and-freshly isolated fetal human epiphyses accurately mimic the normal pattern of gene expression of articular cartilage.
2. HMSCs treated with IGF-1, as well as the isolated human epiphyses grown in the joint simulator, mimic the processes relevant to cartilage rehabilitation, i.e., normal chondrocyte maturation and excessive synthesis of high quality matrix (for HMSC) and preservation of normal cartilage architecture (for epiphyses). Note: to mimic cartilage rehabilitation following injury, a cartilage defect was inflicted in some of the epiphyses grown in the joint simulator.

3. FGF-2 and IL-1β are both osteogenic factors that augment osteogenic initiation of HMSBCs.

4. Mechanical stimulation in the form of stretch or compression may cause cartilage defects that lead to degradation of the surrounding cartilage and enhanced superoxide anion and nitric oxide (NO) synthesis, thereby mimicking the OA phenotype.

Therefore, changes in gene expression caused by treatment with IL1 or FGF2 and/or mechanical stress may be connected to OA development and, therefore, represent potential targets for drug intervention.

In Vitro Model Systems

Human Mesenchymal Stem Cells (HMSCs). The pathogenesis of OA suggests that in diseased articular cartilage, the repair processes are perturbed in the sense that mesenchymal stem cells start to differentiate not only into the chondrocyte (i.e., normal) lineage but also into osteogenic and fibrogenic (i.e., abnormal) lineages. In addition, articular chondrocytes that are usually arrested at the stage of collagen type II differentiate further, to the stage of hypertrophic chondrocytes. This, in turn, may also augment the osteoblastic and osteoclastic (these precursors are not the resident ones and are generated within bone marrow) differentiation of progenitor cells by secretion of osteogenic factors. According to pre-existing knowledge, several insults may cause the stimulation of mesenchymal stem cells and their abnormal differentiation. These include extensive changes in the composition and organization of the ECM, secretion of growth factors, cytokines, chemokines and continuous mechanical constraint. Along these lines, the identification and characterization of intracellular signaling pathways activated by different stimuli in mesenchymal stem cells represent a mandatory step. For this reason, in vitro studies are carried out in a model of adult human mesenchymal stem cell cultures, derived directly from articular cartilage.

Treatment with the following was studied:
IGF-1: growth factor that is beneficial for normal cartilage function and rehabilitation. Inhibits the final differentiation of chondrocytes and cartilage vascularization that finally leads to its replacement by bone.
Interleukin-1: the inflammatory cytokine, known to be overproduced in OA joints. Induces expression of cartilage-degrading enzymes and bone-resorptive cytokines and bioactive molecules, like TNF-α, IL-6, soluble IL-6 receptor (sIL-6R) and NO.
FGF-2: fibroblast growth factors have been implicated in the pathogenesis of OA and animal models of this disease. Severe OA patients showed significantly higher FGF-2 concentrations than mild OA patients. Osteoclastogenesis in a co-culture system that was stimulated by the synovial fluid of severe RA patients was significantly inhibited by a neutralizing antibody against FGF-2, and this inhibition was stronger than that of antibodies against other cytokines. The inventors concluded that the increase in endogenous FGF-2 levels in the synovial fluid of OA patients may play a role in joint destruction by inducing the osteoblast and osteoclast lineages.
Mechanical stimulation: the constant mechanical load on a joint is one of the leading causes of OA development.
   Ex Vivo Model Systems
   Entire Isolated Epiphyses Grown in Joint Simulator

As described above.

### EXAMPLE 2

Establishment of Experimental Model Systems

In Vitro Experiments

A series of in vitro experiments using HMSCs was conducted. The cells were exposed to a panel of treatments which cause either chondrogenic (e.g., IGF-1) or osteogenic and angiogenic (e.g., IL1 and FGF-2) responses. Two rounds of in vitro experiments were performed. Initially, a calibration experiment was carried out in order to determine the time/dose kinetics of the cellular response to the stimuli. Based on this study, a large-scale experiment was performed from which RNA is prepared and used in the course of the gene expression profiling experiments.

Calibration of In Vitro Cell System

In order to validate the differentiation response of the HMSCs to the various stimuli, cells exposed to different treatments were tested for the expression of markers specific for osteoblastic and chondroblastic lineages by immunohistochemistry and staining procedures. To optimize the response of HMSCs to differentiation treatment, the study was performed on cells grown at varying densities (sparse and confluent).

TABLE 1
Treatment regimes of HMSC applied in the pilot study

| | Duration of treatment | | | | | | |
|---|---|---|---|---|---|---|---|
| | | | | | 48 | 3 | 6 |
| Type of treatment | 0 | 1 hr | 12 hrs | 24 hrs | hrs | days | days |
| No Treatment | + | | + | + | + | + | + |
| IL-1β 0.5 ng/ml | | | + | + | + | + | + |
| and 10 ng/ml | | | | | | | |
| IGF-1 10 ng/ml | | | + | + | + | + | + |
| and 20 ng/ml | | | | | | | |
| FGF-2 10 ng/ml | | | + | + | + | + | + |
| and 100 ng/ml | | | | | | | |
| Mechanical stress* | | + | | | | | |
| (compression & | | | | | | | |
| stretching) | | | | | | | |

*as described in Experimental Procedures

The following differentiation markers were tested:

Markers of Chondrocyte Terminal Differentiation and of Osteogenesis:

Osteocalcin (osteocytes)
VEGF (hypertrophic chondrocytes, angiogenesis)
Fibroblast growth factor receptor 3 (FGFR 3) (chondrocytic progenitors and upper hypertrophic chondrocytes)
   Markers of Chondrogenesis:
Collagen type II (chondrocyte maturation)
Alcian Blue (chondrocyte maturation)

The results obtained indicate the following:
1. HMSCs grew and matured under high density (confluent) conditions and displayed some signs of differentiation into both chondrogenic and osteogenic lineages without, however, marked expression of markers specific for hypertrophic chondrocytes and osteocytes
2. Treatment with IGF-1 accelerated chondrocyte maturation and cartilage matrix production as demonstrated by Alcian Blue staining, by increased expression of collagen type II and decreased expression of FGFR3.
3. Treatment with either IL-1, or FGF-2 seemed to have a complex effect on HMSCs: the treatment had the following effects:
(i) induced proliferation of progenitor cells as indicated by increased FGFR-3 expression (may also reflect the induction of terminal chondrocyte differentiation, see below);
a) promoted terminal chondrocyte differentiation and angiogenesis as indicated by increased VEGF expression;
b) stimulated osteogenesis as indicated by increased expression of osteocalcin;
c) inhibited normal cartilage matrix production as indicated by decreased staining with Alcian Blue.

### Conclusions

Treatment of HMSCs with IGF-1 stimulated only the chondrogenic lineage whereas osteoblastic differentiation was somehow inhibited. Therefore, it can serve as a model system for discovery of genes, the products of which either promote normal chondrogenesis or inhibit the generation of osteophytes in OA.

Treatment of HMSCs with IL-1β or with FGF-2 reproduced most of the aspects of OA. Therefore those genes, the expression of which is affected by both treatments, may serve as potential targets for development of anti-OA drugs.

Full Scale In Vitro Experiments for Gene Expression Profiling

Based on the results of the pilot study the following adjustments were made:
1. Only a single cytokine/growth factor dose in each treatment was used, since no dose dependence of HMSC response was observed. This observation can be explained by the fact that concentrations of cytokine/growth factors used in this study are above the physiological range. Thus, the maximal response could already be achieved by treatment with any of the proposed doses and their further increase did not augment the cell response. In addition, treatment with high doses of IL-1β appeared cytotoxic.
2. The number of time points at which cells are harvested for RNA preparation was increased significantly to allow for the increased resolution of development of genetic response to the treatments.

The summary of the treatment regimes used for the full-scale experiment is shown in Table 2.

TABLE 2
Treatment regimes of HMSC applied in the full-scale study

| | Duration of treatment | | | | | | |
|---|---|---|---|---|---|---|---|
| | | | | | | 3 | 6 |
| Type of treatment | 0 | 1 hr | 12 hrs | 24 hrs | 48 hrs | days | days |
| No Treatment | + | | + | + | + | + | + |
| IL-1β 0.5 ng/ml | | | + | + | + | + | + |
| IGF-1 10 ng/ml | | | + | + | + | + | + |
| FGF-2 10 ng/ml | | | + | + | + | + | + |
| Mechanical stress | | + | | | | | |
| (compression) | | | | | | | |
| Mechanical stress | | + | | | | | |
| (tension) | | | | | | | |

At each time point, ALP staining was performed. Each experiment was carried out twice, yielding 44 experiments (22 treatment regimes×2 repetitions). The RNA from these experiments was used for the preparation of the dedicated OA chip (see below) and for the generation of probes for hybridization to the “OA” chip and to the “Fibrosis” chip.

Ex Vivo Models for OA Study

In addition to the in vitro experiments in which HMSC were employed, RNA was extracted from fetal (22 week-old) human epiphyses directly upon removal and after the 3 days growth in a joint simulator. To trigger the processes of matrix rehabilitation, some of the epiphyses were injured prior to their positioning into the joint simulator. This treatment replaced that previously proposed where the epiphyses were to be grown ex vivo under regular tissue culture conditions. This was because the pilot experiment demonstrated that the tissues failed to survive under such conditions. Each of the ex vivo experiments was carried out twice yielding a total of 6 experiments (i.e., 3 conditions×2 repetitions) (see Table 3). The RNA from the ex vivo experiments was also used for the preparation of the dedicated “OA” chip. The generated probes were hybridized to the “OA” chip and to the “Fibrosis” chip.

TABLE 3
Treatment regimes of fetal human epiphyses in the joint simulator

| Treatment | Number of epiphyses |
|---|---|
| Freshly isolated | 6 (3 per hybridization)* |
| Grown in joint simulator for 3 days | 6 (3 per hybridization) |
| Injured prior to growth in joint simulator | 6 (3 per hybridization) |
| for 3 days | |

*Each probe was prepared from RNA pools extracted from 3 similarly treated epiphyses.

### EXAMPLE 3

Identification of Genes Involved in the Development of OA and Cartilage Rehabilitation and Screening Candidate Drugs for the Treatment Thereof.

Preparation of the “OA” and “Fibrosis” Chips

The “OA” chip was prepared from the pool of RNA's extracted from HMSC treated as described above as well as from RNA obtained from the ex vivo experiments, by co-applicant's SDGI method, described in PCT Patent Application Publication No. WO 01/75180, fully incorporated herein by reference. It contains a total of 10,000 cDNA clones. Also the Fibrosis” chip was prepared by applicant's said SDGI method.

Hybridizations to cDNA Microarrays

Probe Labeling and Hybridization to DNA Microarrays

cDNA probes were synthesized from 1 μg of polyA RNA derived from every sample using reverse transcriptase (Superscript, Gibco-BRL) and 18-mer oligo-dT primer. Cy3-dCTP (Amersham) or Cy5-dCTP (Amersham) were incorporated during the RT reaction, to label the cDNA. Hybridization, subsequent scanning, and visualization were performed as previously described (Schena, M. et al. (1996) Proc Natl Acad Sci USA 93, 10614-10619). The quality control of hybridizations was performed according to applicant's methods.

The Hybridization Scheme

cDNA probes were hybridized to the OA and to the Fibrosis human cDNA microarrays according to the scheme presented in Table 3.

Probe 2 in all hybridizations was an identical probe comprised of a pool of zero time point RNA extractions (Table 4, Common control). This served as a common normalizing probe and allowed comparison of results from different hybridizations in formal statistical analysis. Probe 1 in each hybridization was prepared from RNA extracted from cells or ex vivo organ cultures cultivated under the defined treatment conditions. The entire hybridization set was repeated twice, yielding a total of 26 hybridization types×2 cDNA microarrays×2 repetitions=104 hybridizations.

TABLE 4
Hybridization scheme

| Probe | | | Human articular cartilage |
|---|---|---|---|
| No. | Probe Name | Dye | RNA, Treatment. |
| 1 | SOA1 | cy5 | Control 12 h |
| | NML21 (Common control) | cy3 | Normal |
| 2 | SOA2 | cy5 | Control 24 h |
| | NML21 (Common control) | cy3 | Normal |
| 3 | SOA3 | cy5 | Control 48 h |
| | NML21 (Common control) | cy3 | Normal |
| 4 | SOA4 | cy5 | Control 3 days |
| | NML21 (Common control) | cy3 | Normal |
| 5 | SOA5* | cy5 | Control 6 days |
| | NML21 (Common control) | cy3 | Normal |
| 6 | SOA6 | cy5 | IL-1b 12 h |
| | NML21 (Common control) | cy3 | Normal |
| 7 | SOA7 | cy5 | IL-1b 24 h |
| | NML21 (Common control) | cy3 | Normal |
| 8 | SOA8 | cy5 | IL-1b 48 h |
| | NML21 (Common control) | cy3 | Normal |
| 9 | SOA9 | cy5 | IL-1b 3 days |
| | NML21 (Common control) | cy3 | Normal |
| 10 | SOA10 | cy5 | IL-1b 6 days |
| | NML21 (Common control) | cy3 | Normal |
| 11 | SOA11 | cy5 | IGF-1 12 h |
| | NML21 (Common control) | cy3 | Normal |
| 12 | SOA12 | cy5 | IGF-1 24 h |
| | NML21 (Common control) | cy3 | Normal |
| 13 | SOA13 | cy5 | IGF-1 48 h |
| | NML21 (Common control) | cy3 | Normal |
| 14 | SOA14 | cy5 | IGF-1 3 days |
| | NML21 (Common control) | cy3 | Normal |
| 15 | SOA15 | cy5 | IGF-1 6 days |
| | NML21 (Common control) | cy3 | Normal |
| 16 | SOA16 | cy5 | FGF-2 12 h |
| | NML21 (Common control) | cy3 | Normal |
| 17 | SOA17 | cy5 | FGF-2 24 h |
| | NML21 (Common control) | cy3 | Normal |
| 18 | SOA18 | cy5 | FGF-2 48 h |
| | NML21 (Common control) | cy3 | Normal |
| 19 | SOA19 | cy5 | FGF-2 3 days |
| | NML21 (Common control) | cy3 | Normal |
| 20 | SOA20 | cy5 | FGF-2 6 days |
| | NML21 (Common control) | cy3 | Normal |
| 21 | OA23 | cy5 | Joint simulator with injury |
| | | | 3 days |
| | NML21 (Common control) | cy3 | Normal |
| 22 | OA24 | cy5 | Joint simulator 3 days control |
| | NML21 (Common control) | cy3 | Normal |
| 23 | OA27 | cy5 | Freshly isolated epiphyses |
| | NML21 (Common control) | cy3 | Normal |
| 24 | SOA30 | cy5 | HMSC |
| | NML21 (Common control) | cy3 | Normal |
| 25 | SOA31 | cy5 | 17 weeks AC 1 hr |
| | | | compression (MF) |
| | NML21 (Common control) | cy3 | Normal |
| 26 | SOA32 | cy5 | 17 weeks AC 1 h tension |
| | | | (MF) |
| | NML21 (Common control) | cy3 | Normal |

*Probe SOA5 has displayed artifactual behavior and was therefore removed from further analysis.

Analysis of Hybridization Results

The hybridization data were analyzed using an algorithm for quality control and two different algorithms for gene clustering.

Results

After performing the full analysis of hybridization results the inventors ended up with a list of genes, from which 57 genes were derived from the “Fibrosis” chip and 197 genes were derived from the “OA” chip.

Some of the known genes were analyzed in the light of information available from biomedical literature and from public databases.

IL-1- and FGF-regulated genes were of major interest because of their potential implication in the osteoarthritic phenotype (see above), i.e., degradation of cartilage matrix and stimulation of ectopic bone formation.

One of the identified genes that was up-regulated by IL1-β, with previously unknown involvement in arthritic diseases, was found to be ISLR, having the nucleotide sequence as presented in SEQ ID NO. 1, which encodes a polypeptide, the sequence of which is as presented in SEQ ID NO:2.

In addition, in ex vivo experiments, the otherwise observed up-regulation of this gene was suppressed when embryo epiphyses were cultivated in joint simulator.

Here for the first time the inventors demonstrate up-regulation of ISLR by treatment of HMSC cells with IL1-β that may have an osteogenic effect Therefore, ISLR is a preferred target for screening candidate drugs for the treatment of OA, i.e., for identifying and isolating compounds which inhibit or stimulate the gene transcription or translation or the protein expression or activity of ISLR. ISLR is also a preferred marker for the diagnosis of OA and for monitoring the progression of OA, in both the presence and absence of treatment.

ISLR may also be a target for screening candidate drugs for the treatment of OP, fractures and other bone disorders i.e., for identifying and isolating compounds which inhibit or stimulate the gene transcription or translation or the protein expression or activity of ISLR. ISLR may also be a marker for the diagnosis of OP and for monitoring the progression of OP and fracture healing, in both the presence and absence of treatment.

### EXAMPLE 4

Characteristics of Human ISLR Gene and Gene Product, and Human ISLR Expression in Human Articular Cartilage

ISLR (Genomic Location: 15q23-q24) is a known human protein containing both a LRR and an immunoglobulin (Ig)-like domain (Nagasawa A et al. (1997) Genomics 44(3):273-279; Nagasawa A et al. (1999) Genomics 61(1):37-43). This gene was referred to as ISLR, to stand for “Immunoglobulin Superfamily-containing LRR”. The predicted protein comprises 428 amino acids, and comprises the following putative domains (see FIG. 1):
a) A cleavable, well-defined N-terminal signal peptide at amino acid residues 1-19,
b) a leucine-rich repeat (LRR) region (amino acid residues 18-230). This region can be divided into N-terminal and C-terminal domains of LRR (amino acid residues 18-54 and 180-230, respectively). Between them, there are five LRR (amino acid residues 50-72, 73-96, 97-120, 121-144, 145-168),
c) one immunoglobulin C-2 type repeat at amino acid positions 248-334.

Despite the variety of functions, all members of the Ig superfamily are involved in adhesion or binding to other proteins in solution or at the cell surface. Therefore it is possible that the ISLR protein may also interact with other proteins or cells.

Northern blot analysis has previously shown that a 2.4 kb ISLR transcript is present in various human tissues, including retina, heart, skeletal muscle, prostate, ovary, small intestine, thyroid, adrenal cortex, testis, stomach, and spinal cord, as well as fetal lung and fetal kidney (Nagasawa et al. (1997) Genomics 44(3):273-279; Nagasawa et al. (1999) Genomics 61(1):37-43).

The amino acid homology between ISLR amino acid residues 1-428 and human OCP amino acid residues 1-551 (end of 1^{st} Ig domain from the N-terminus) is 19%, and between ISLR amino acid residues 1428 and human Adlican amino acid residues 1-562 (end of 1^{st} Ig domain from the N-terminus) is 20% (FIG. 2). Despite the relatively low level of homology at the amino acid level, structural similarity among the four molecules (OCP, Adlican, Adlican-2 and ISLR) is striking, with high conservation of several structural domains (FIG. 1).

The inventors have found that ISLR expression was affected in human articular cartilage by various treatments. Compared to untreated human articular cartilage controls, expression of OCP, to which ISLR is structurally homologous, was down-regulated in the joint simulator and also under mechanical stress conditions, and remained unaffected by IGF treatment and by FAD (FGF-2+ascorbic acid+dexamethasone) treatment. In contrast, expression of ISLR was up-regulated by IL1-β treatment when compared to controls.

### EXAMPLE 5

It Situ Hybridization: Methods and Results Obtained

The pattern of gene expression is studied by in situ hybridization on sections of bones from OA patients, OA and RA animal models and from human embryonic tissue. Menisectomy in rats is used as an OA model. Collagen induced arthritis (CIA) is used as a RA model in mice. Human samples are obtained during surgery from patients with clinically diagnosed osteoarthritis (OA). These samples contain regions with normal cartilage as well as OA cartilage. For in situ analysis, tibia, together with the respective knee joint, is excised. Bones are fixed for three days in 4% paraformaldehyde and then decalcified for four days in a solution containing 5% formic acid and 10% formalin. Decalcified bones are postfixed in 10% formalin for three days and embedded in paraffin.

Six m sections are prepared and hybridized in situ. After hybridization, sections are dipped into nuclear track emulsion and exposed for three weeks at 4° C. Autoradiographs are developed, stained with hematoxylin-eosin and studied under microscopy using brightfield and darkfield illumination.

Results of In Situ Hybridization with ISLR Gene

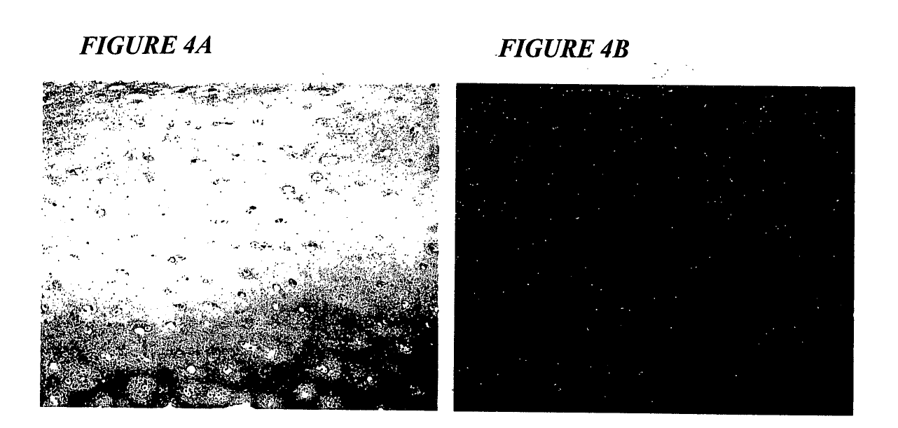

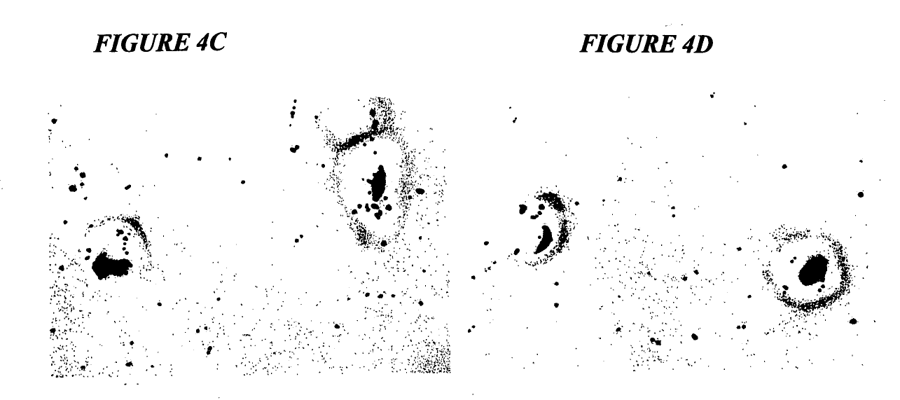

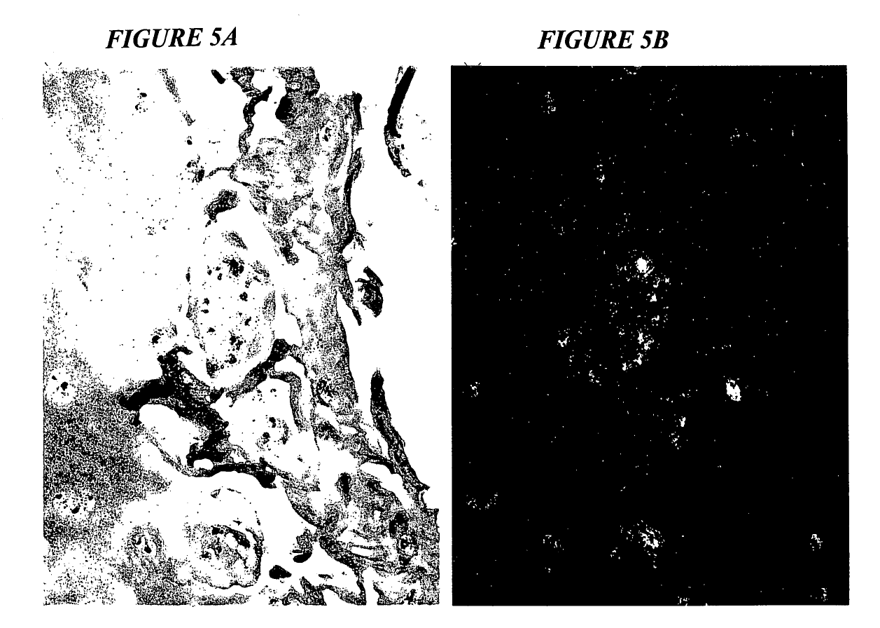

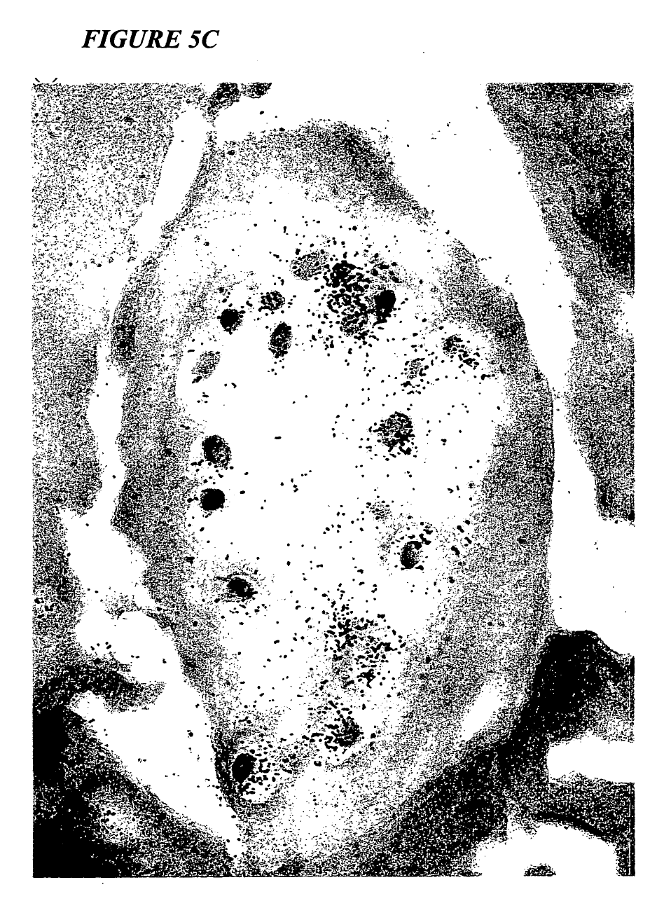

A. For further assessment of cell and tissue specificity of ISLR gene expression in bone development, an in situ hybridization study was performed on sections of multitissue block containing multiple samples of adult human tissue, obtained during surgery. Human samples were obtained during surgery from patients with clinically diagnosed osteoarthritis (OA). These samples contained regions with normal cartilage as well as OA cartilage. In situ hybridization was performed, and S³⁵ labeled riboprobes gave high sensitivity and reasonable microscopic resolution. In these samples it was found that there was little or no expression of ISLR in intact articular cartilage, while there was expression in synovial cells in both normal and osteoarthritic samples. There was a prominent signal in activated chondrocytes in eroded cartilage. These results are clearly shown in FIGS. 4 and 5.

B. Collagen-induced arthritis (CIA) in mice was produced essentially as described by Trentham et al (Trentham D. E, Townes A. S, Kang A. H (1977)). DBA/1 mice were subjected to collagen type II injections for 21-50 days following which swelling and joint appearance was monitored. Serial sectioning of knee and ankle joint samples was performed, the sections were stained with HE (hematoeosin) and the sections were studied and a series of sections suitable for the ISH was selected.

In situ analysis in intact mouse cartilage showed some expression in osteoprogenitor cells and weak expression in both synovial and chodrocyte cells. In the pathological CIA derived samples, prominent expression was observed in the hyperplastic synovial and bone marrow cells, specifically in the pannus cells.

### EXAMPLE 6

Experimental Procedures Used for Investigating the ISLR Gene

Cell Culture: the Following Cell Lines are Used;

HMSC—Normal adult articular cartilage progenitor cells are obtained from human cartilage. The cartilage is dissected and cultured in fresh DMEM medium supplemented with 10% FCS, L-glutamine and antibiotics. Two weeks after the cultivation of dissected cartilage, the remaining pieces are removed and the attached cells are used.

U20S—The human osteoblast-like osteosarcoma cell line is obtained from the American Type Culture Collection (HTB-96).

RCJ3.1C5.18—This cell line is described in Grigoriadis A. E, Heersche J. N, Aubin J E (1996) Analysis of chondroprogenitor frequency and cartilage differentiation in novel family of clonal chondrogenic rat cell lines, Differentiation, 60:299-307.

293—These human kidney cells are obtained from the American Type Culture Collection (CRL-1573).

Mechanical Stress

HMSCs are grown in a culture flask to confluence. Secondary subcultures are seeded at a density of 1×10⁵ cell/cm² onto flexible polyurethane membranes, which are attached by clamps to a mechanical device. For better cell attachment and growth, the membranes are pretreated with complete serum for 60 minutes at room temperature. The cells are allowed to adhere to the membranes during 24 hrs. The cultures undergoing a tension treatment are stretched by moving the clamps (causing up to 33% stretching). The tension is kept constant for 1 hour. The cells are collected by mechanical scraping and used for RNA isolation. Compression treatments are performed as follows: The flexible membranes are attached to the mechanical device by clamps and stretched prior to cell seeding by 25% of their original length, then cells are seeded and incubated for adherence for 24 hrs. After 24 hrs, the strain is released and the membranes gain back their original length, forming the compression. This compression is kept constant for 1 hour prior to RNA extraction, which is performed as described above.

Transfection Stable transfection—RCJ3.1C5.18 or C3H10T1/2 cells are transfected with the human ISLR gene cloned into the pCMVNSVneo expression vector using Lipofectamine 2000 reagent (GibcoBRL) according to the manufacturer's instructions. Stable clones are selected after the addition of 0.3 mg G418/ml medium. RT-PCR.

Total RNA is extracted from stable RCJ3.1C5.18 or C3H10T1/2 clones using the EZ-RNA isolation kit (Biological Industries) according to the manufacturer's protocol. First strand cDNA synthesis and PCR reaction are performed using the Superscript II kit (GibcoBRL) according to the manufacturer's instructions.

Northern Blot Analysis

Northern blot analysis of human ISLR is performed using RNA extracted from U2OS cells. A blot containing 2 μg of poly(A)+ RNA is probed with the whole ORF of ISLR. The protocol is described in “Current protocols in molecular biology”, Ausubel F M et al. (ed), (1987), Vol.1, Section4.9.1.

Ex Vivo Model System

Entire Isolated Epiphyses Grown in Joint Simulator

In addition to in vitro experiments, the inventors also use an ex vivo model of isolated fetal human epiphyses grown in a joint simulator, as described above.
   Glucosaminoglycan Marker Staining

1. Alcian blue—Embryonic bone is fixed with Bouin fixative for 10 minutes, stained with Alcian Blue, which stains cartilage matrix deposition, (1% in 3% acetic acid) for 30 min and washed with distilled water.

2. Alizarin red—Embryonic bone is fixed in 70% ethanol solution, incubated for 60 minutes on ice and stained with 40 mM alizarin red solution, which stains calcified tissue, (Sigma) for 10 minutes.

Proliferation Examination

PCNA—Immunohistochemistry with anti-PCNA antibodies (DAKO) is performed on sections of embryonic bone according to the manufacturer's instructions.

Hypertrophy Analysis

Collagen type X—Immunohistochemistry with anti-collagen type X (quartett) is performed on sections of embryonic bone according to the manufacturer's instructions.

Osteoblastic Markers

Alcian phosphatase staining, osteocalcein immunohistochemistry

Osteoclastic Markers

TRAP in situ hybridization

Animal Models

1. Collagen-Induced Arthritis (CIA)

CIA in mice (see results above) is described in Trentham D. E, Townes A. S, Kang A. H (1977). Autoimmunity to type II collagen: an experimental model of arthritis. J. Exp. Med. 146: 857-868.

2. Adjuvant-Induced Arthritis (AA)

AA is described in Kong Y Y et al., (1999). Activated T cells regulate bone loss and joint destruction in adjuvant arthritis through osteoprotegerin ligand, Nature, 402:304-308.

3. Menisectomy Model

Menisectomy is described in Han F, Ishiguro N, Ito T, Sakai T, Iwata H. (1999). Effects of sodium hyaluronate on experimental OA in rabbit knee joints. Nagoya J Med Sci November, 62(3-4):115-126.

4. Ovariactomy (ovx) Model

ISLR is administered to 4-month-old rats following bilateral ovx to evaluate the extent of bone formation rate in osteoporotic rats.

### EXAMPLE 7

Evaluation of ISLR Modulators as Potential Drugs for OA

Selection of ISLR as a target for screening candidate modulators as potential drugs for inhibiting or delaying OA was based on its up-regulation upon IL1-β treatment, as disclosed herein.

Evaluation of ISLR Inhibitors as Potential Drugs for OA

In order to evaluate the applicability of different ISLR modulators as potential drugs for OA, the different modulators are examined for their ability to lead to inhibition or attenuation of chondrocyte proliferation and terminal differentiation, as well as to inhibition of development of arthritis. The different modulators are examined using the following evaluation test systems:

In Vitro Test System

Subcloning of the Human ISLR Full-Length cDNA

The entire ORF of human ISLR is described as follows:

Accession number AB024537 [Homo sapiens cell line: FLEB14-14 DNA, clone lib: Keio BAC library clone: KB116G11]; Nagasawa A et al., Genomics 1997, 44(3):273-279; Nagasawa A et al., Genomics 1999, 61(1):37-43.

The full ISLR ORF, with a Flag-Tag at the N-terminus, was subcloned into the pCMVNSV-neo and into pBScKS for TNT reaction. An additional expression vector was prepared by subcloning this gene into the pIRES-puro expression vector (Clontech).

Examination of ISLR Constructs by Transient Transfection of 293 Cells

In order to examine the constructs, transient transfection of 293 cells with the human ISLR cDNA (containing Flag-tag and cloned into pIRES puro) is performed. Cells are grown in serum-free medium for two days and the media and lysates are collected. Proteins are separated on 10% SDS gel followed by blotting. Membrane is reacted with anti-Flag antibodies.

Purification of Polypeptide and Verification of Activity

The desired construct, comprising the functional portion of ISLR, is expressed in 293 T cells. Western blot analysis of the medium, using antibody to the Flag-tag, demonstrates the presence of the desired polypeptide. This polypeptide is purified from the medium, using a column of anti-Flag-tag antibodies. This purified polypeptide is added at a concentration of 200 ng/ml to the mesenchymal cell line C3H10T1/2. Seven days post-administration, the cultures are checked for cartilage/bone nodule formation. Osteoblastic and chondrogenic differentiation is determined using alizarin red staining and Alcian blue, respectively.

Enhanced Proliferation or Differentiation Rate in Transfected Cells

Different end-point parameters indicating inhibition of chondrocyte proliferation and terminal differentiation by a test ISLR inhibitor are examined using various transfected cell lines (e.g., chondrocytes, endothelial cells) over-expressing exogenous human ISLR gene. Higher proliferation or differentiation rate indicates that the said gene is possibly involved in pathways that induce arthritis.

Establishment of Stable Transfected Cell Line

The human ISLR cDNA lacking the Flag-tag is cloned into the pCMVneo expression vector, and is used for stable transfection of the RCJ3.1.C5.18 or C3H10T1/2 cell line. More than 30 clones are isolated and screened using RT-PCR. The highest expressing clones are selected for drug evaluation experiments.

The effect of ISLR over-expression is examined using different end point parameters indicating chondrocyte/osteoblast proliferation, chondrocyte/osteoblast differentiation, and osteoclastogenesis. These parameters have previously been shown to be related to OA. The iii vitro test system of the invention is used as well for evaluation of the ability of different ISLR inhibitors to inhibit and attenuate these effects. Differentiation of chondrocytes is evaluated by Alcian Blue staining, ALP activity and collagen type X immunohistochemical staining. Increase in ALP activity or in collagen type X immunohistochemical staining indicates final differentiation of chondrocytes.

Differentiation of osteoprogenitor cells is evaluated by ALP activity. Proliferation of chondrocytes/osteoblasts is evaluated by measuring cell number and thymidine incorporation.

Ex Vivo Test System

The effect of ISLR or ISLR modulators on cartilage and bone formation is examined ex vivo using embryonic bone organ culture. Hind legs are obtained from mouse embryo (E16). Bone cultures are performed in α-modified Eagle's medium supplemented with 10% fetal calf serum (FCS), 0.05 mg/ml ascorbic acid, and 1 mM β-glycerolphosphate. One bone per well is cultured in 300 μl complete medium in a 24-well tissue culture plate, in a 5% CO₂ incubator at 37° C. and 98% humidity. Increasing concentrations of recombinant ISLR or of different ISLR inhibitors are added to the culture medium and bones are cultured for 7 days. Bones treated with the polypeptide or its inhibitor, as well as control bones, are evaluated at time 0 and after 7 days of culture.

Sections prepared from control and treated bones are stained using alizarin red and Alcian Blue for evaluating chondrocyte final differentiation. Longitudinal growth of the bone is calculated as the increase in length of the hypertrophic region and the calcified bone. Hypertrophy is further evaluated using typeX collagen staining. Proliferation is examined by PCNA.

In Vivo Test System

The effect of ISLR or of different ISLR modulators on different parameters related to OA such as chondrocyte proliferation, terminal differentiation and development of arthritis, is evaluated using two in vivo systems; one system over-expresses exogenous ISLR and the other expresses endogenous ISLR. Both test systems are contemplated by the present invention as a model for OA.

Transgenic Mice Expressing Exogenous ISLR

The effect of ISLR over-expression is examined using different end point parameters indicating chondrocyte proliferation, chondrocyte final differentiation, and osteoclastogenesis, and development of arthritis.

Transgenic FVBN mice expressing human ISLR cDNA under collagen type II promoter/enhancer are established. Cartilage development is examined in sections obtained from transgenic mouse embryos (E17) or from 1-week-old mice. Evaluation of final differentiation is performed by staining sections with Alcian Blue and alizarin red. Evaluation of proliferation is performed by PCNA.

The development of arthritis is examined in adult ISLR transgenic mice by examining paw thickness and articular cartilage histology.

Arthritic Rats Expressing Endogenous ISLR

The arthritic rat model is used as an in vivo evaluating system expressing endogenous ISLR.

Collagen arthritis is induced in Female Lewis rats according to the method of Trentham discussed above. The emulsion is prepared by adding 1.6 mg/ml of bovine collagen type II and 0.4 mg/ml of adjuvant peptide solution to an equal volume of Freund's incomplete adjuvant (DIFCO, MI) and stirring with an homogenizer for 15 minutes, 4° C. at 10,000 rpm. On day 0, each animal receives intradermally 0.8 mg of collagen in 1 ml emulsion.

The effect of ISLR modulation by different candidate inhibitors is evaluated in this system using different end-point parameters indicating chondrocyte proliferation, chondrocyte differentiation, bone formation, osteoclastogenesis, and development of arthritis.

Different ISLR modulators can be administered to arthritic rats (collagen type II-induced arthritis (CIA)). The modulators are dissolved in water and administered orally or directly into the joint of the arthritic rats. The development of arthritis is monitored by measuring paw thickness. In addition, histological examination is performed on sections obtained from treated and control animals.

Evaluation of differentiation is performed by staining sections with Alcian Blue and alizarin red. Evaluation of proliferation is performed by PCNA.

### EXAMPLE 8

Additional Possible Investigations:

A. ISLR Induction During Mesenchymal Cell Differentiation Towards Osteogenesis

Mesenchymal stem cells (USC) are multipotent, self-renewing cell populations which undergo differentiation and commitment to give rise to monopotent cells of specified lineages, such as osteoblasts. The mechanisms of commitment and self-renewal are not fully understood, but may be regulated by factors such as Bone Morphogenetic Proteins (BMPs), differentiation factors such as retinoic acid and steroid hormones such as glucocorticoids. Furthermore, BMP and retinoic acid act synergistically to stimulate osteoblastic commitment and cell proliferation.

In order to determine whether ISLR expression is induced upon osteoblastic commitment, quiescent C3H10T1/2 murine MSC cultures are stimulated with BMP and RA for 24 hours and cultured in full medium for a further 3 days. RNA is extracted from non-treated cells as well as from cells harvested at 24 hrs, 48 hrs and 72 hrs after the beginning of the treatment, and is used for RT-PCR analysis with ISLR-specific primers. In parallel, cells are stained for ALP to determine osteoblastic commitment.

B. Switch of Pre-Myoblasts to Osteoblasts

Pre-myoblastic cells (C2C12) give rise to mature myoblasts. As with C3H10T1/2, the administration of BMP and RA to these cells can induce osteoblastic differentiation. To investigate the expression pattern of ISLR during this differentiation switch we introduce BMP and RA to C2C12 cells and analyze cell fate and expression pattern (as described above for C3H10T1/2 cells).

These assays may demonstrate the involvement of ISLR in the early stages of osteogenesis.

Having thus described in detail preferred embodiments of the present invention, it is to be understood that the invention defined by the appended claims is not to be limited by particular details set forth in the above description as many apparent variations thereof are possible without departing from the spirit or scope thereof.

1-36. (canceled)

37. A method of treatment of arthritis in a subject in need of such treatment comprising administering to the subject an effective amount of an antisense oligonucleotide which inhibits expression of the ISLR gene.

38. The method of claim 37 wherein the arthritis is osteoarthritis.

39. The method of claim 37 wherein the arthritis is rheumatoid arthritis.

40. The method of claim 37 wherein the antisense oligonucleotide is an RNA oligonucleotide.

41. The method of claim 38 wherein the antisense oligonucleotide is an RNA oligonucleotide.

42. The method of claim 39 wherein the antisense oligonucleotide is an RNA oligonucleotide.

43. A method of treatment of bone disease in a subject in need of such treatment comprising administering to the subject an effective amount of an antisense oligonucleotide which inhibits expression of the ISLR gene.

44. The method of claim 43 wherein the bone disease is osteopenia.

45. The method of claim 43 wherein the bone disease is osteoporosis.

46. The method of claim 43 wherein the antisense oligonucleotide is an RNA oligonucleotide.

47. The method of claim 43 wherein the bone disease is selected from the group consisting of periodontosis, osteopetrosis, osteosarcoma, chondrosarcoma, or osteosclerosis.

48. The method of claim 47 wherein the antisense oligonucleotide is an RNA oligonucleotide.

49. The method of claim 43 wherein the treatment of bone disease comprises promotion of bone building.

50. The method of claim 49 wherein the bone building comprises bone fracture healing.

51. The method of claim 49 wherein the bone building comprises bone elongation.

52. The method of claim 49 wherein the antisense oligonucleotide is an RNA oligonucleotide.

53. The method of claim 50 wherein the antisense oligonucleotide is an RNA oligonucleotide.

54. The method of claim 51 wherein the antisense oligonucleotide is an RNA oligonucleotide.

55. A pharmaceutical composition comprising an antisense oligonucleotide which inhibits expression of the ISLR gene and a pharmaceutically acceptable carrier.

56. The pharmaceutical composition of claim 55 wherein the antisense oligonucleotide is an RNA oligonucleotide.


SEQUENCE LISTING (4 sequences)

SEQ ID NO: 1 - DNA, 1284 bp, Homo sapiens
atgcaggagctgcatctgctctggtgggcgcttctcctgggcctggctcaggcctgccctgagccctgcgactgtggggaaaagtatggcttccagatcgccgactgtgcctaccgcgacctagaatccgtgccgcctggcttcccggccaatgtgactacactgagcctgtcagccaaccggctgccaggcttgccggagggtgccttcagggaggtgcccctgctgcagtcgctgtggctggcacacaatgagatccgcacggtggccgccggagccctggcctctctgagccatctcaagagcctggacctcagccacaatctcatctctgactttgcctggagcgacctgcacaacctcagtgccctccaattgctcaagatggacagcaacgagctgaccttcatcccccgcgacgccttccgcagcctccgtgctctgcgctcgctgcaactcaaccacaaccgcttgcacacattggccgagggcaccttcaccccgctcaccgcgctgtcccacctgcagatcaacgagaaccccttcgactgcacctgcggcatcgtgtggctcaagacatgggccctgaccacggccgtgtccatcccggagcaggacaacatcgcctgcacctcaccccatgtgctcaagggtacgccgctgagccgcctgccgccactgccatgctcggcgccctcagtgcagctcagctaccaacccagccaggatggtgccgagctgcggcctggttttgtgctggcactgcactgtgatgtggacgggcagccggcccctcagcttcactggcacatccagatacccagtggcattgtggagatcaccagccccaacgtgggcactgatgggcgtgccctgcctggcacccctgtggccagctcccagccgcgcttccaggcctttgccaatggcagcctgcttatccccgactttggcaagctggaggaaggcacctacagctgcctggccaccaatgagctgggcagtgctgagagctcagtggacgtggcactggccacgcccggtgagggtggtgaggacacactggggcgcaggttccatggcaaagcggttgagggaaagggctgctatacggttgacaacgaggtgcagccatcagggccggaggacaatgtggtcatcatctacctcagccgtgctgggaaccctgaggctgcagtcgcagaaggggtccctgggcagctgcccccaggcctgctcctgctgggccaaagcctcctcctcttcttcttcctcacctccttc

SEQ ID NO: 2 - protein, 428 aa, Homo sapiens
MQELHLLWWALLLGLAQACPEPCDCGEKYGFQIADCAYRDLESVPPGFPANVTTLSLSANRLPGLPEGAFREVPLLQSLWLAHNEIRTVAAGALASLSHLKSLDLSHNLISDFAWSDLHNLSALQLLKMDSNELTFIPRDAFRSLRALRSLQLNHNRLHTLAEGTFTPLTALSHLQINENPFDCTCGIVWLKTWALTTAVSIPEQDNIACTSPHVLKGTPLSRLPPLPCSAPSVQLSYQPSQDGAELRPGFVLALHCDVDGQPAPQLHWHIQIPSGIVEITSPNVGTDGRALPGTPVASSQPRFQAFANGSLLIPDFGKLEEGTYSCLATNELGSAESSVDVALATPGEGGEDTLGRRFHGKAVEGKGCYTVDNEVQPSGPEDNVVIIYLSRAGNPEAAVAEGVPGQLPPGLLLLGQSLLLFFFLTSF

SEQ ID NO: 3 - protein, 562 aa, homo sapiens
MPKRAHWGALSVVLILLWGHPRVALACPHPCACYVPSEVHCTFRSLASVPAGIARHVERINLGFNSIQALSETSFAGLTKLELLMIHGNEIPSIPDGALRDLSSLQVFKFSYNKLRVITGQTLQGLSNLMRLHIDHNKIEFIHPQAFNGLTSLRLLHLEGNLLHQLHPSTFSTFTFLDYFRLSTIRHLYLAENMVRTLPASMLRNMPLLENLYLQGNPWTCDCEMRWFLEWDAKSRGILKCKKDKAYEGGQLCAMCFSPKKLYKHEIHKLKDMTCLKPSIESPLRQNRSRSIEEEQEQEEDGGSQLILEKFQLPQWSISLNMTDEHGNMVNLVCDIKKPMDVYKIHLNQTDPPDIDINATVALDFECPMTRENYEKLWKLIAYYSEVPVKLHRELMLSKDPRVSYQYRQDADEEALYYTGVRAQILAEPEWVMQPSIDIQLNRRQSTAKKVLLSYYTQYSQTISTKDTRQARGRSWVMIEPSGAVQRDQTVLEGGPCQLSCNVKASESPSIFWVLPDGSILKAPMDDPDSKFSILSSGWLRIKSMEPSDSGLYQCIAQVRDE

SEQ ID NO: 4 - protein, 390 aa, Homo sapiens
MPKRAHWGALSVVLILLWGHPRVALACPHPCACYVPSEVHCTFRSLASVPAGIAKHVERINLGFGILKCKKDKAYEGGQLCAMCFSPKKLYKHEIHKLKDLTCLKPSIESPLRQNRSRSIEEEQKQEENGDSQLILEKIQLPQWSISLNMTDEHGNLVNLVCDIKKPMDVYKIHLNQTDPPDIDINAMVALDFEYPMTQENYENLWKLIAYYSEVPMKLHRELMLSKHPRVSYQYRQDADEEALYYTGVRAQILAEPEWIMQPSIDIQLNRPQSTAKKVLLSYYNQYSQTIATKDTRQARGRSWVMIEPSRAVQKDQTVLEGGRCQLSCNVKASESPSIFWVLPDGSILKVPVDDPDSKFSILSSGWLRIKSMEPSDSGLYQCIAQVRDE